SEQUENCE LISTING (48 sequences)

SEQ ID NO: 1 - protein, 5 aa, Artificial Sequence
  Description of Artificial Sequence Synthetic peptide
  MOD_RES (3)..(3): Any amino acid
LPXTG

SEQ ID NO: 2 - protein, 5 aa, Artificial Sequence
  Description of Artificial Sequence Synthetic peptide
LPKTG

SEQ ID NO: 3 - protein, 5 aa, Artificial Sequence
  Description of Artificial Sequence Synthetic peptide
LPATG

SEQ ID NO: 4 - protein, 5 aa, Artificial Sequence
  Description of Artificial Sequence Synthetic peptide
LPNTG

SEQ ID NO: 5 - protein, 5 aa, Artificial Sequence
  Description of Artificial Sequence Synthetic peptide
LPETG

SEQ ID NO: 6 - protein, 5 aa, Artificial Sequence
  Description of Artificial Sequence Synthetic peptide
  MOD_RES (3)..(3): Any amino acid
LPXAG

SEQ ID NO: 7 - protein, 5 aa, Artificial Sequence
  Description of Artificial Sequence Synthetic peptide
LPNAG

SEQ ID NO: 8 - protein, 5 aa, Artificial Sequence
  Description of Artificial Sequence Synthetic peptide
  MOD_RES (3)..(3): Any amino acid
LPXTA

SEQ ID NO: 9 - protein, 5 aa, Artificial Sequence
  Description of Artificial Sequence Synthetic peptide
LPNTA

SEQ ID NO: 10 - protein, 5 aa, Artificial Sequence
  Description of Artificial Sequence Synthetic peptide
  MOD_RES (3)..(3): Any amino acid
LGXTG

SEQ ID NO: 11 - protein, 5 aa, Artificial Sequence
  Description of Artificial Sequence Synthetic peptide
LGATG

SEQ ID NO: 12 - protein, 5 aa, Artificial Sequence
  Description of Artificial Sequence Synthetic peptide
  MOD_RES (3)..(3): Any amino acid
IPXTG

SEQ ID NO: 13 - protein, 5 aa, Artificial Sequence
  Description of Artificial Sequence Synthetic peptide
IPNTG

SEQ ID NO: 14 - protein, 5 aa, Artificial Sequence
  Description of Artificial Sequence Synthetic peptide
IPETG

SEQ ID NO: 15 - protein, 5 aa, Artificial Sequence
  Description of Artificial Sequence Synthetic peptide
NPQTN

SEQ ID NO: 16 - protein, 5 aa, Artificial Sequence
  Description of Artificial Sequence Synthetic peptide
  MOD_RES (3)..(3): Any amino acid
LAXTG

SEQ ID NO: 17 - protein, 5 aa, Artificial Sequence
  Description of Artificial Sequence Synthetic peptide
  MOD_RES (3)..(3): Any amino acid
LPXSG

SEQ ID NO: 18 - protein, 5 aa, Artificial Sequence
  Description of Artificial Sequence Synthetic peptide
LSETG

SEQ ID NO: 19 - protein, 5 aa, Artificial Sequence
  Description of Artificial Sequence Synthetic peptide
  MOD_RES (3)..(3): Any amino acid
LPXCG

SEQ ID NO: 20 - protein, 5 aa, Artificial Sequence
  Description of Artificial Sequence Synthetic peptide
  MOD_RES (3)..(3): Any amino acid
LPXAG

SEQ ID NO: 21 - protein, 5 aa, Artificial Sequence
  Description of Artificial Sequence Synthetic peptide
  MOD_RES (1)..(1): Any amino acid
XPETG

SEQ ID NO: 22 - protein, 61 aa, Staphylococcus aureus
VDNKFNKEQQNAFYEILHLPNLNEEQRNAFIQSLKDDPSQSANLLAEAKKLNDAQAPKMRM

SEQ ID NO: 23 - protein, 56 aa, Unknown
  Description of Unknown Streptococcal Protein G sequence
MTFKLIINGKTLKGEITIEAVDAAEAEKIFKQYANDYGIDGEWTYDDATKTFTVTE

SEQ ID NO: 24 - protein, 148 aa, Staphylococcus aureus
MQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 25 - protein, 148 aa, Artificial Sequence
  Description of Artificial Sequence Synthetic polypeptide
MQAKPQIPKDKSKVAGYIEIPDADIKEPVYPGPATPEQLNRGVSFAEENESLDDQNISIAGHTFIDRPKYQFTNLKAAKKGSMVYFKVGNETRKYKMTSIRDVKPTDVEVLDEQKGKDKQLTLITCDDYNEKTGVWEKRKIFVATEVK

SEQ ID NO: 26 - protein, 148 aa, Artificial Sequence
  Description of Artificial Sequence Synthetic polypeptide
MQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMSSIRDVKPTDVEVLEEQKGKEKQLTLITCDDYNEKTGVWEKRKIFVATEVK

SEQ ID NO: 27 - protein, 148 aa, Artificial Sequence
  Description of Artificial Sequence Synthetic polypeptide
MQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVK

SEQ ID NO: 28 - protein, 5 aa, Artificial Sequence
  Description of Artificial Sequence Synthetic peptide
LLQGA

SEQ ID NO: 29 - protein, 14 aa, Artificial Sequence
  Description of Artificial Sequence Synthetic peptide
KQSGPGLVQPSQSL

SEQ ID NO: 30 - protein, 14 aa, Artificial Sequence
  Description of Artificial Sequence Synthetic peptide
VTVSAASTKGPSVF

SEQ ID NO: 31 - protein, 16 aa, Artificial Sequence
  Description of Artificial Sequence Synthetic peptide
PLAPSSKSTSGGTAAL

SEQ ID NO: 32 - protein, 17 aa, Artificial Sequence
  Description of Artificial Sequence Synthetic peptide
PLAPSSKSTSGGTAALF

SEQ ID NO: 33 - protein, 13 aa, Artificial Sequence
  Description of Artificial Sequence Synthetic peptide
DGVEVHNAKTKPR

SEQ ID NO: 34 - protein, 9 aa, Artificial Sequence
  Description of Artificial Sequence Synthetic peptide
HNHYTQKSL

SEQ ID NO: 35 - protein, 21 aa, Artificial Sequence
  Description of Artificial Sequence Synthetic peptide
VYACEVTHQGLSSPVTKSFNR

SEQ ID NO: 36 - protein, 23 aa, Artificial Sequence
  Description of Artificial Sequence Synthetic peptide
VYACEVTHQGLSSPVTKSFNRGE

SEQ ID NO: 37 - protein, 6 aa, Artificial Sequence
  Description of Artificial Sequence Synthetic peptide
GGSGGS

SEQ ID NO: 38 - protein, 4 aa, Artificial Sequence
  Description of Artificial Sequence Synthetic peptide
LPET

SEQ ID NO: 39 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 7 aa, Artificial Sequence
  Description of Artificial Sequence Synthetic peptide
LPETGGG

SEQ ID NO: 46 - protein, 16 aa, Artificial Sequence
  Description of Artificial Sequence Synthetic peptide
  MOD_RES (7)..(7): Fluorescently labeled with TAMRA
PLAPSSKSTSGGTAAL

SEQ ID NO: 47 - protein, 4 aa, Artificial Sequence
  Description of Artificial Sequence Synthetic peptide
RRGW

SEQ ID NO: 48 - protein, 7 aa, Artificial Sequence
  Description of Artificial Sequence Synthetic peptide
KHRFNKD

(19) United States
(12) Patent Application Publication
(10) Pub. No.: US 2024/0229000 A1
(43) Pub. Date: Jul. 11, 2024

(54) METHODS AND COMPOSITIONS THEREOF FOR SITE-SPECIFIC LABELING OF HUMAN IgG BY PROXIMITY-BASED SORTASE-MEDIATED LIGATION

(71) Applicants: THE TRUSTEES OF THE UNIVERSITY OF PENNSYLVANIA, Philadelphia, PA (US); AlphaThera LLC, Philadelphia, PA (US)

(72) Inventors: Andrew TSOURKAS, Bryn Mawr, PA (US); Wendy YU, West Windsor, NJ (US); Wendy Yu, San Mateo, CA (US); Per-Åke Nygren, EKERÖ (SE)

(73) Assignees: THE TRUSTEES OF THE UNIVERSITY OF PENNSYLVANIA, Philadelphia, PA (US); AlphaThera LLC, Philadelphia, PA (US)

(21) Appl. No.: 18/559,333

(22) PCT Filed: May 6, 2022
(86) PCT No.: PCT/US22/28134

§ 371 Date: Nov. 6, 2023

Related U.S. Application Data
(60) Provisional application No. 63/185,843, filed on May 7, 2021.

(51) Int. Cl. C12N 9/52; C07K 14/315; C07K 16/22; C07K 16/32

(52) U.S. Cl. CPC C12N 9/52; C07K 14/315; C07K 16/22; C07K 16/32; C12Y 304/2207; C07K 2319/70

(57) ABSTRACT

The present disclosure provides compositions and methods for site-specific labeling of antibodies by proximity-based sortase-mediated ligation. The ligation method utilizes a non-canonical isopeptide ligation reaction catalyzed by newly identified variants of S. aureus sortase A. An antibody binding domain (e.g., protein A or protein G) is fused to the variants of SrtA to bring the enzyme into close proximity of an antibody, thereby significantly increases the efficiency of isopeptide bond formation.

### STATEMENT REGARDING FEDERALLY SPONSORED RESEARCH

This invention was made with government support under Grant Number CA221374 awarded by the National Institutes of Health. The United States government has certain rights in the invention.

### FIELD OF THE INVENTION

This disclosure relates in general to the field of antibody conjugation technology Described herein are compositions and methods for labeling of antibodies by proximity-based sortase-mediated ligation.

### BACKGROUND OF THE INVENTION

Antibody-drug conjugates (ADCs) utilize the specificity of antibodies to deliver highly potent cytotoxic drugs to cells (e.g., cancer cells) in a targeted fashion. In general, ADCs include a monoclonal antibody specific to a target antigen that is typically overexpressed or uniquely expressed on a target cell. A potent drug that is often not systemically well-tolerated on its own is conjugated to the antibody through a linker. With this design, ADCs can deliver the drug to the target cells by first binding to a target antigen expressed on the cell surface. Upon binding, the antigen-ADC complex is internalized via receptor-mediated endocytosis and then eventually gets trafficked into lysosomes. At this stage, ADCs are degraded within the lysosomes, which releases free drug into the cell and induces a therapeutic effect (e.g., cell death) through different mechanisms depending on the payload type. Currently, the main drug payloads used for ADC conjugation include DNA-damaging agents and microtubule inhibitors. In addition, ADCs can induce cell death via the bystander effect where dying cells release free drug into the surrounding microenvironment, which can kill surrounding cells. By targeting delivery of drugs (e.g. cytotoxins) to cells, ADCs improve the drugs' efficacy, while reducing off-target toxicities to normal tissues. In turn, ADCs increase the therapeutic index (ratio of maximum tolerated dose to minimum effective dose) compared to that of standard chemotherapy, which is severely limited by its high toxicity.

While ADCs have demonstrated potential in cancer treatment, there has been little clinical success. To date, only four ADCs have been approved by the FDA out of the 200+ ADC programs that have undergone clinical trials so far. In large part, ADCs have had poor clinical success due to major weaknesses in its design, which have reduced its therapeutic potential. One critical shortcoming involves the tact that conventional conjugation methods for attaching linker-drug moieties to an antibody yield heterogeneous mixtures of ADCs with variable drug-to-antibody ratios (DAR). Traditionally, ADC conjugation utilizes solvent-accessible, reactive amino acid residues, such as lysines and cysteines. In lysine conjugation, lysines are randomly acylated with activated esters, which results in 0-8 drug molecules per antibody. One study identified that at least 40 lysine residues out of the 86 present on the heavy and light chains are modified during conjugation, generating a mixture of over a million unique ADC species with different drug loads and conjugation sites. In the case of cysteine conjugation, interchain disulfide bonds are reduced to expose eight thiol groups, which are randomly alkylated with maleimides. This also results in a DAR that ranges from 0-8, which produces a mixture of over a hundred unique ADC species. Consequently, this heterogeneity poses significant problems in the clinic, since each unique ADC species will display unpredictable in vivo pharmacokinetic, safety, and efficacy profiles. Moreover, consistency in batch-to-batch production of ADCs is difficult to achieve. Therefore, in recent years, there has been a shift towards site-specific conjugation where the DAR and linker-drug conjugation sites are precisely controlled in order to produce more homogeneous and potent ADCs.

Several methods for conjugating drug payloads at defined sites to produce site-specific ADCs have been investigated. In one approach, engineered cysteine residues are introduced into the antibody for site-specific labeling with thiol-reactive linkers. This approach is predicated on the fact that the engineered cysteine residues are substituted at sites that do not disrupt antibody structure and function. In order to determine optimal cysteine substitution positions, a method called Phage ELISA for Selection of Reactive Thiols (PHESELECTOR) was developed. In this assay, reactive cysteine residues are introduced into the antibody, which are then displayed using phage technology and screened to select for thiol groups that do not interfere with antibody binding. ADCs produced from these optimal engineered cysteine sites are termed THIOMAB drug conjugates (TDCs). Using PHESELECTOR, Junutula et al. (2008) developed anti-MUC16 TDCs with a DAR of 2 at high purity and found that conjugation was specific to the engineered cysteine residues. Compared to ADCs produced from conventional cysteine conjugation, the TDCs were equally as efficacious in both in vitro and in vivo studies despite having a lower average DAR. Additionally, the TDCs exhibited a greater therapeutic index since they were better tolerated at high doses in animal studies.

A second site-specific approach that has been developed focuses on glycoconjugation using enzymes, such as glycotransferases and transglutaminases. Glycotransferases are naturally involved in oligosaccharide synthesis, particularly in transferring sugar moieties from an activated glycosyl donor to a nucleophilic glycosyl acceptor. In this platform, a glycotransferase, such as β1,4-Galactosyltransferase 1 (Gal-T1), is mutated at its catalytic pocket site to increase its sugar donor specificity. This allows the mutant glycotransferase to attach chemically active sugar residues, such as C2-keto-Gal, to any lipid or protein with a glycosylation site. In doing so, any molecule with a bioorthogonal reactive group can be conjugated via the chemical handle on the sugar moiety. In the case of antibodies, human IgGs have a conserved N-glycosylation site at the asparagine 297 residue on the Fc chain, making it a highly attractive target site. In one study. Boeggeman et al. (2009) site-specifically conjugated trastuzumab with Alexa Fluor 488 C₅-aminooxyacetamide by degalactosylating the N-glycans attached to asparagine 297 down to the G0 glycoform. This enabled the transfer of C2-keto-Gal using Gal-T1, which facilitated conjugation at that site. Another enzyme that has been explored is transglutaminase, which catalyzes the formation of isopeptide bonds between glutamine side chains and primary amine groups. In initial studies, Jeger et al. (2010) found that glutamine 295, once deglycosylated, could be used as a substrate for transglutaminase. Using this method, Dennler et al. (2014) modified trastuzumab with an azido-PEG-amine and then reacted the antibody with cyclooctyne-functionalized MMAE to successfully produce uniform ADCs with a DAR of 2.

In addition to glycoconjugation, ribosomal incorporation of unnatural amino acids (UAAs) has been explored as a site-specific conjugation method. One UAA of interest is p-acetylphenylalanine (pAcPhe), which can be conjugated at its keto group using alkoxy-amine-modified drugs via oxime ligation. In order to incorporate UAAs, such as pAcPhe, the amber stop codon, UAG, and tRNA/aminoacyl-tRNA synthetase (aaRS) pair are most widely used. Essentially. UAG is inserted at defined sites in the gene encoding the desired protein, which is expressed in cells, along with aaRS to facilitate pAcPhe incorporation at the UAG site (Liu and Schultz, 2010). Other unnatural amino acids used for engineering site-specific ADCs include selenocysteine, azido-lysine, and azido-methyl-phenylalanine (Hallam et al., 2015).

Lastly, short peptide tags can be employed to enzymatically generate site-specific ADCs. As described previously, transglutaminases catalyze isopeptide bonds between glutamine residues and primary amine groups. Instead of conjugating at glutamine 295 which requires a deglycosylation step first, one group engineered the glutamine tag, LLQGA, to different sites on anti-Her2 and anti-M ISI antibodies. Due to the fact that it recognizes the glutamine tag, but not native glutamine residues, transglutaminase (mT) from Streptoverticillium mobaraense was used to attach monomethyl dolastatin 10 (MMAD) at the glutamine tag sites. Through this method, Strop et al. (2013) produced ADCs with intact binding affinities, effective in vitro and in vivo activity, and improved tolerability at high doses compared to ADCs generated from conventional cysteine conjugation. In addition, in rat studies, the clearance rate and linker stability of the ADCs were found to be dependent on the conjugation site. In particular, ADCs conjugated at the heavy chain were cleared faster than antibody alone and had a DAR of 1 despite starting out with a DAR of 1.8-1.9, while ADCs conjugated at the light chain remained intact and were cleared at a similar rate as antibody alone.

Another enzymatic conjugation method utilizes Staphylococcus aureus sortase A (SrtA), which is a calcium-assisted transpeptidase that covalently anchors proteins to the peptidoglycan cell wall of Gram-positive bacteria. Specifically, SrtA recognizes the amino acid motif, LPXTG (SEQ ID NO: 1, X is any amino acid), which can be engineered into a protein. The active cysteine on SrtA cleaves between the threonine and glycine residues to form a thioester acyl-enzyme intermediate. Upon nucleophilic attack by an N-terminal oligoglycine peptide, an amide bond is formed between the carboxyl group of threonine and the α-amine of glycine, and SrtA is released from the tagged protein. In turn, a compound modified with an N-terminal oligoglycine peptide can be theoretically ligated onto a protein with the LPXTG motif via SrtA-mediated transpeptidation. With this technology, several groups have site-specifically attached a variety of different compounds, such as drug payloads and imaging agents, onto antibodies and other proteins. ADCs produced using SrtA were also found to be potent in both in vitro and in vivo studies with complete tumor regression and no toxicities observed in rodent xenograft models.

Overall, site-specific conjugation has proven to vastly enhance the safety and anti-tumor efficacy of ADCs compared to those derived from conventional conjugation techniques. Outside of glycoengineering, the primary advantage of most site-specific methods is the fact that the number and location of conjugation sites can be exactly controlled and defined. However, a majority of these approaches require modifications to the antibody, which can negatively impact the structure, function, and stability of the antibody. In addition, other disadvantages of current site-specific methods include inefficient drug conjugation, poor product yield, and unstable antibody-drug linker attachment.

Accordingly, there exists a need to develop new methods for the efficient labeling of antibodies that do not negatively impact antibody production, impair antibody function, or require large excesses of material for labeling, which is inefficient and costly.

### SUMMARY OF THE INVENTION

In one aspect, described herein are near traceless, sortase-mediated conjugation methods that allow region-specific modification of native IgG without the need for genetic engineering or glycan modification. These methods use a non-canonical isopeptide ligation reaction catalyzed by newly identified variants of S. aureus sortase A, Srt A (N127K or T156S/D176E/D170E), to label antibodies. An antibody binding domain is fused to the variants of SrtA to bring the enzyme into close proximity to IgG, thereby significantly increasing the efficiency of isopeptide bond formation.

In one aspect, provided herein are compositions comprising: an antibody-binding domain fused to a transpeptidase. In one embodiment, the antibody binding domain is Protein G. In another embodiment, the antibody binding domain is a subdomain of Protein G or variant thereof, including but not limited to the hyperthermally stable variant of Protein G, HTB1. In yet another embodiment, the antibody binding domain is Protein A. In another embodiment, the antibody binding domain is a subdomain of Protein A or variant thereof, including but not limited to Protein Z or a calcium-sensitive derivative of Protein Z. In one embodiment, the transpeptidase is a sortase. In one embodiment, the sortase is Sortase A or variant thereof. In one embodiment, the sortase includes three point mutations, T156S/D176E/D170E (referred to as 3M SrtA). In yet another embodiment, the sortase includes a single point mutation, N127K (referred to as 1M SrtA).

In one aspect, provided herein are methods of producing an antibody conjugate, the methods comprising the steps of: (a) binding an antibody-binding domain-transpeptidase fusion protein to an antibody; (b) linking a peptide to the antibody via proximity-based sortase-mediated isopeptide ligation (PBS-IL), wherein the peptide contains a sortase recognition motif and an isopeptide bond is formed between a lysine on the antibody and the peptide. In one embodiment, the peptide is labeled with cargo, which includes but is not limited to fluorescent dyes, haptens (e.g. biotin), polymers, contrast agents (e.g. gadolinium, radionuclides), chelated metals, therapeutic agents, sensitizers, oligonucleotides, or combinations thereof. In one embodiment, the peptide is further conjugated or fused to additional protein or peptide sequences.

In one aspect, the invention provided herein are methods of producing an antibody conjugate, the methods comprising the steps of: (a) binding an antibody-binding domain-transpeptidase fusion protein to an antibody, wherein the antibody has been engineered with a sortase recognition motif near the c-terminus of its heavy and/or light chains; (b) linking a peptide to the antibody via proximity-based sortase-mediated protein ligation (PBS-PL), wherein the peptide possesses an N-terminal glycine and a peptide bond is formed between the peptide and the sortase recognition motif on the antibody. In one embodiment, the peptide is labeled with cargo, which includes but is not limited to fluorescent dyes, haptens (e.g. biotin), polymers, contrast agents (e.g. gadolinium, radionuclides), chelated metals, therapeutic agents, sensitizers, oligonucleotides, or combinations thereof. In one embodiment, the peptide is further conjugated or fused to additional protein or peptide sequences.

Also provided herein are nucleic acids and vectors that encode the foregoing antibody-binding domain-transpeptidase fusion protein. Further provided herein are cells that express the foregoing antibody-binding domain-transpeptidase fusion protein.

Other features and advantages of the present invention will become apparent from the following detailed description, examples, and figures. It should be understood, however, that the detailed description and specific examples, while indicating preferred embodiments of the invention, are given by way of illustration only, since various changes and modifications within the spirit and scope of the invention will become apparent to those skilled in the art from this detailed description.

These and other aspects of the invention will be appreciated from the ensuing descriptions of the figures and detailed description of the invention.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

FIG. 1 illustrates proximity-based sortase-mediated isopeptide ligation (PBS-IL). SrtA-pG or SrtA-pZ binds to the Fc region of the antibody. Cargo (e.g., a drug, fluorophore, biotin, PEG, etc., represented by a star) is modified with the SrtA recognition motif, LPETG. In the presence of Ca²⁺ and LPETG-modified cargo, SrtA facilitates isopeptide ligation between the LPETG-modified cargo and a proximal Fc lysine residue to generate an antibody conjugate. When a calcium-dependent pZ derivative is used, washing with EDTA (5 mM) buffer allows efficient removal of Srt-pZ. Otherwise, an acidic buffer can be used to dissociate pG/pZ from antibody.

FIGS. 2A-2B show evaluation of labeling efficiencies of different SrtA variants on Cetuximab. FIG. 2A shows degree of TAMRA labeling was compared for WT/5M SrtA vs. WT/5M SrtA-pG vs. 1 M SrtA-pG to determine benefits of proximity-based SrtA labeling via Protein G and the evolved point mutation on SrtA (1M). FIG. 2B shows labeling efficiencies of evolved mutants, 3M and 1M SrtA, fused to Protein G or Protein Z. Reaction conditions for all labeling experiments were as follows: 2 μg Cetuximab, 200 μM TAMRA-LPETG, 500 μM CaCl₂), and 1.25 μM SrtA variant in 10 mM Tris-HCl buffer (37° C. overnight). Labeling efficiencies were calculated by taking the UV/SDS-PAGE ratio of the fluorescent intensity band to the heavy chain protein band intensity. For each experiment, labeling efficiencies were normalized against the maximum UV/SDS ratio to compare across reaction conditions. One-way analysis of variance (ANOVA) was used to determine statistical significance (*P<0.05, n.s.=not significant), followed by Tukey's multiple comparisons test. Bar graphs are represented as average±SD. N=3 per reaction condition.

FIGS. 3A-3B show optimization of the isopeptide ligation reaction. Labeling reactions for Cetuximab were carried out by titrating the 1M SrtA-pZ concentration from 0.5-3.75 μM (FIG. 3A) (corresponding to SrtA/antibody molar ratios ranging from 0.4:1 to 3:1) and the TAMRA-LPETG concentration from 25-400 μM (FIG. 3B). Other reaction components were held constant: 2 μg Cetuximab, 500 μM CaCl₂), 200 μM TAMRA-LPETG (for 1M SrtA-pZ titration) and 1.25 μM 1M SrtA-pZ (for TAMRA-LPETG titration) in 10 mM Tris-HCl buffer (37° C. overnight). Labeling efficiencies were calculated by taking the UV/SDS-PAGE ratio of the fluorescent intensity band to the heavy chain protein band intensity. For each experiment, labeling efficiencies were normalized against the maximum UV/SDS-PAGE ratio to compare across reaction conditions. One-way ANOVA was used to determine statistical significance (*P<0.05, n.s.=not significant), followed by Tukey's multiple comparisons test. Bar graphs are represented as average±SD. N=3 per reaction condition.

FIGS. 4A-4D show LC-MS analyses of native Cetuximab (FIG. 4A) and Cetuximab-TAMRA (FIG. 4B), following proximity-based sortase-mediated ligation. Comparison of samples pre- and post-labeling show that the light chain (LC) is modified with up to one TAMRA molecule and the heavy chains (HC) is modified with 1 or 2 TAMRA molecules. Shown are representative LC-MS analyses after alkylation and digestion with chymotrypsin of a peptide fragment of Cetuximab (FIG. 4C) or Cetuximab-TAMRA (FIG. 4D). The expected native, unlabeled peptide is found in the Cetuximab, but not the Cetuximab-TAMRA sample. Conversely, a TAMRA-labelled peptide is found following digestion of Cetuximab-TAMRA, but not Cetuximab. Antibodies were reduced and deglycosylated for all LC-MS studies.

FIGS. 5A-5B show determination of average drug-to-antibody ratio (DAR) via UV/Vis spectroscopy. Absorption spectra for Cetuximab (FIG. 5A) and Cetuximab-Ahx-TAMRA (FIG. 5B) were measured using Nanodrop. Protein and TAMRA have absorption maxima at 280 and 555 nm, respectively. Using Beer-Lambert law for a multi-component system, individual concentrations of TAMRA and Cetuximab in the Cetuximab-Ahx-TAMRA sample were calculated from the A₂₈₀ and A₅₅₅ of TAMRA-LPETG, Cetuximab, and Cetuximab-Ahx-TAMRA. The average DAR was determined to be 2.32, which was found by dividing the molar concentration of TAMRA with that of Cetuximab.

FIGS. 6A-6N show LC-HRMS analyses of peptides from heavy- and light-chain cetuximab after chymotrypsin, trypsin, Asp-N, or Glu-C digestion. FIG. 6A shows MH₂²⁺ (upper) and MH₃³⁺ (middle) chromatograms from heavy-chain chymotrypsin peptide K⁵QSGPGLVQPSQSL {retention time (r.t.)=32.1 min}. No response was observed in the chromatogram from MH₃³⁺ of the corresponding TAMRA-LPETG-adduct at a r.t. of 38.6 min (lower). FIG. 6B shows reduction of signals from MH₂²⁺ (upper, 70%) and MH₃³⁺ (middle, 68%) in chromatograms from heavy-chain chymotrypsin peptide K⁵QSGPGLVQPSQSL (r.t.=32.2 min) after conjugation with TAMRA-LPETG. MH₃³⁺ of the corresponding TAMRA-LPETG-adduct was observed in the chromatogram at a r.t. 38.6-min (lower). FIG. 6C shows MH₂²⁺ (upper) and MH₃³⁺ (middle) chromatograms from heavy-chain chymotrypsin peptide VTVSAASTK¹²³GPSVF (r.t.=33.5 min). No response was observed in the chromatogram from MH₃³⁺ of the corresponding TAMRA-LPETG-adduct at a r.t. of 38.1 min (lower). FIG. 6D shows reduction of signals from MH₂²⁺ (upper, 84%) and MH₃³⁺ (middle, 75%) in chromatograms from heavy-chain chymotrypsin peptide VTVSAASTK¹²³GPSVF (r.t.=33.6 min) after conjugation with TAMRA-LPETG. MH₃³⁺ of the corresponding TAMRA-LPETG-adduct was observed in the chromatogram at a r.t. of 38.1-min (lower). FIG. 6E shows MH₂²⁺ (upper) chromatogram from heavy-chain chymotrypsin peptide PLAPSSK¹³⁵STSGGTAAL (r.t.=29.9 min). No response was observed in the chromatogram for MH₃³⁺ of the corresponding TAMRA-LPETG-adduct at a r.t. of 36.6 min from (lower). FIG. 6F shows reduction of signal in the chromatogram from MH₂²⁺ (upper, 90%) for heavy-chain chymotrypsin peptide PLAPSSK¹³⁵STSGGTAAL F (r.t.=29.9 min) after conjugation with TAMRA-LPETG. MH₃³⁺ of the corresponding TAMRA-LPETG-adduct was observed in the chromatogram at a r.t. of 36.6-min (lower). FIG. 6G shows MH₃³⁺ (upper) chromatogram from heavy-chain Asp-N peptide DGVEVHNAKTK²⁹²PR (r.t.=25.1 min). No response was observed in the chromatogram from MH₃³⁺ from the corresponding TAMRA-LPETG-adduct at a r.t. of 36.9 min (lower). FIG. 6H shows reduction of signal from MH₃³⁺ (upper, 57%) in the chromatogram from heavy-chain Asp-N peptide DGVEVHNAKTK²⁹²PR (r.t.=25.2 min) after conjugation with TAMRA-LPETG. MH₃³⁺ of the corresponding TAMRA-LPETG-adduct was observed in the chromatogram at a r.t. of 36.9 min (lower). FIG. 6I shows MH₂²⁺ (upper) chromatogram from heavy-chain chymotrypsin peptide HNHYTQK⁴⁴¹SL (r.t.=33.9 min). No response was observed in the chromatogram from MH₃³⁺ of the corresponding TAMRA-LPETG-adduct at a r.t. of 26.1 min (lower). FIG. 6J shows reduction of signals from MH₂²⁺ (upper, 62%) in the chromatogram of heavy-chain chymotrypsin peptide HNHYTQK⁴⁴¹SL (r.t.=33.6 min) after conjugation with TAMRA-LPETG. MH₃³⁺ of the corresponding TAMRA-LPETG-adduct was observed in the chromatogram at a r.t. of 26.1-min (lower). FIG. 6K shows MH₂²⁺ (upper) and MH₃³⁺ (lower) chromatograms from light-chain trypsin peptide VYACEVTHQGLSSPVTK²⁰⁷SFNR (r.t.=32.7 min). No response was observed from in the chromatogram for MH₃³⁺ of the corresponding TAMRA-LPETG-adduct at a r.t. of 39.6 min (lower). FIG. 6L shows reduction of signals from MH₂²⁺ (upper, 75%) and MH₃³⁺ (middle, 95%) in chromatograms from light-chain trypsin peptide VYACEVTHQGLSSPVTK²⁰⁷SFNR (r.t.=32.7 min) after conjugation with TAMRA-LPETG. MH₄⁴⁺ of the corresponding TAMRA-LPETG-adduct was observed in the chromatogram at a r.t. of 39.6-min (lower). FIG. 6M shows MH₃³⁺ (upper) and MH₄⁴⁺ (middle) chromatograms from light-chain Glu-C peptide VYACEVTHQGLSSPVTK²⁰⁷SFNRGE (r.t.=30.1 min). No response was observed in the chromatogram from MH₄⁴⁺ of the corresponding TAMRA-LPETG-adduct at a r.t. of 36.6 min (lower). FIG. 6N shows reduction of signals from MH₃³⁺ (upper, 100%) and MH₄₄ (middle, 98%) in chromatograms from light-chain Glu-C peptide VYACEVTHQGLSSPVTK²⁰⁷SFNRGE (r.t.=30.1 min) after conjugation with TAMRA-LPETG. MH₄⁴⁺ of the corresponding TAMRA-LPETG-adduct was observed in the chromatogram at a r.t. of 36.6-min (lower).

FIG. 7 shows a model of the three-dimensional structure of Cetuximab with Protein Z (green) bound at the CH₂-CH₃ hinge region. The heavy chains of Cetuximab are shown in red, while the light chains are shown in blue. The lysine residues that were labeled by proximity-based SrtA-mediated isopeptide ligation are shown in yellow. The PDB structure of Cetuximab is 1YY8 and the PDB structure of Fc and protein Z is 1FC2.

FIGS. 8A-8D show functional binding properties of the SrtA-generated ADC. FIG. 8A shows fluorescence microscopy imaging of labeled EGFR⁺ MDA-MB 468 cells. Fixed MDA-MB 468 cells were treated with either Cetuximab-TAMRA or Cetuximab. Labeled cells were imaged at 40× using bright field illumination and fluorescence. FIG. 8B shows binding affinity of Cetuximab-Ahx-vcMMAE and Cetuximab to MDA-MB 468 cells. Fixed MDA-MB 468 cells were treated with serial dilutions of Cetuximab-Ahx-vcMMAE and Cetuximab, followed by incubation with a PE-goat anti-human secondary antibody. Cell labeling was measured at 544/585 nm. FIG. 8C shows neonatal Fc receptor (FcRn) binding. Cetuximab-Ahx-vcMMAE and Cetuximab were coated onto a 96-well plate to which serial concentrations of biotinylated FcRn (FcGRT+B2M heterodimer) were applied at pH 6.0. After adding the streptavidin-HRP and TMB substrate, FcRn binding was measured by absorbance at 450 nm. FIG. 8D shows Fe-gamma receptor I (FcγRI) binding. The same protocol for FcγRI binding was conducted, but instead, biotinylated FcγRI was applied to the coated plate at neutral pH. The binding affinity, as well as FcRn and FcγRI binding, of Cetuximab is not significantly affected by MMAE conjugation via proximity-based sortase-mediated isopeptide ligation.

FIGS. 9A-9B show in vitro cytolytic activity of the SrtA-generated ADC. The cytolytic activity of Cetuximab-Ahx-vcMMAE was evaluated in two EGFR⁺ cancer cell lines: MDA-MB 468 (FIG. 9A) and A431 (FIG. 9B). The xCELLigence Real-Time Cell Analysis (RTCA) system was used to monitor cytolysis for each cell line. 5000 cells per well were seeded in a 96-well Electronic Microtiter Plate and then treated with Cetuximab-Ahx-vcMMAE, Cetuximab mixed with free MMAE, or Cetuximab only. After 96 hours, cell viability was measured.

FIG. 10 shows amino acid sequences of Cetuximab, Trastuzumab and Rituximab heavy chain (HC) and light chain (LC). All sequences have been aligned. The lysine residues on Cetuximab that were labeled with TAMRA by proximity-based SrtA-mediated isopeptide ligation are highlighted in red, as well as the corresponding lysines on Trastuzumab and Rituximab. The lysine position is noted immediately above the labeled lysine residue. All of the lysine residues that were labeled on Cetuximab are present on Trastuzumab and Rituximab, except for K5. The alternate amino acids at this position are highlighted in yellow.

FIG. 11 shows conjugation of other human IgG antibodies via the near traceless method. Trastuzumab and Rituximab were successfully labeled with TAMRA using either 1M SrtA-pG or 1M SrtA-pZ. Reaction conditions were as follows: 2 μg antibody, 200 μM TAMRA-LPETG, 500 μM CaCl₂), and 1.25 μM 1M SrtA-pG or 1M SrtA-pZ in 10 mM Tris-HCl buffer (37° C. overnight).

FIG. 12 illustrates proximity-based sortase-mediated protein ligation (PBS-PL). A SRM (LPETG) is fused at the c-terminus of the heavy and/or light chain of IgG. An antibody-binding domain (pG or pZ) is then used to bring sortase (Srt) into close proximity of the SRM to efficiently ligate a drug-labeled peptide (GGG). The pG/pZ-Srt is then removed in a Ca2+-free buffer (pZ) or at low pH (pG).

FIGS. 13A-13C show comparison of proximity-based sortase-mediated protein ligation with 1M SrtA versus traditional sortase labeling with the pentamutant SrtA. FIG. 13A: Heavy chain labeling of trastuzumab (HCLPETG+LC) was carried out by titrating 5M SrtA (5M SrtA/antibody molar ratios ranging from 1:1 to 100:1) and comparing the labeling efficiency to that of 5M SrtA-pG, 1M SrtA-pZ, 1M SrtA-pG, and 1M pG-SrtA. The greatest extent of antibody labeling was observed with 1M SrtA-pG, 1M SrtA-pZ, and via traditional Srt labeling when 5M Srt was used at concentrations equivalent to the antibody concentration; however, traditional labeling with 5M Srt A was very sensitive to the concentration of 5M Srt A. Just a two-fold concentration change led to a 25% reduction in ligation efficiency. Moreover, traditional sortase labeling led to significantly higher amounts of an undesirable side product (HC dimers; HCLEPTG dimer), compared with antibodies labeled with 1M Srt-pG or 1M Srt-pZ. A 5M SrtA-pG fusion protein also did not label antibodies as efficiently as 1 M SrtA-pG and 1M SrtA-pZ. Labeling efficiencies of 5M SrtA (FIG. 13B) and 1M SrtA-pZ (FIG. 13C) on the heavy chain were further compared by scaling the GGG-TAMRA concentration from 1-200 μM. Reaction conditions included: 1 μg trastuzumab (HCLPETG+LC), 500 μM CaCl₂, 200 μM GGG-TAMRA, and 0.63 μM SrtA variant (1:1 molar ratio of 1M SrtA-pZ to antibody) in 10 mM Tris-HCl buffer (37° C. overnight). For all titration experiments, all other reactions conditions were held constant. Labeling efficiencies were determined by measuring the fluorescent intensity of the heavy chain band. For each experiment, these values were normalized against the maximum fluorescent intensity to compare across reaction conditions. One-way ANOVA was used to determine statistical significance (*P<0.05, n.s.=not significant), followed by Tukey's multiple comparisons test. Bar graphs are represented as average f SD. N=3 per reaction condition. These results demonstrate that antibody labeling with 1M Srt-pZ can reach completion with ˜3-times less GGG-TAMRA peptide compared with conventional sortase-mediated ligation with 5M SrtA.

FIG. 14 shows different conjugation schemes for IgG using the proximity-based labeling method. Three trastuzumab variants were expressed in which a C-terminal LPETG motif was cloned into the heavy chain only (HCLPETG+LC), the light chain only (HC+LCLPETG), and both the heavy and light chains (HCLPETG+LCLPETG). All variants were labeled under the same conditions: 1 μg trastuzumab variant, 500 μM CaCl₂), 200 μM GGG-TAMRA, and 0.63 μM 1M SrtA-pZ (1:1 molar ratio of 1M SrtA-pZ to antibody) in 10 mM Tris-HCl buffer (37° C. overnight). Correct and efficient labeling of the heavy and/or light chains was observed by UV illumination when samples were analyzed by SDS-PAGE.

FIG. 15 shows heavy chain labeling of trastuzumab (HCLPETG+LC) with WT SrtA-pG, 1M SrtA-pG, and 1M SrtA-pZ was compared to demonstrate that the 1M mutation improves the overall labeling efficiency of sortase. Protein G and Z fused to 1M SrtA were both compared as well since Protein Z offers improved solubility and elution conditions and hence, would be favorable for downstream production and purification of ADCs generated using this approach. The labeling efficiencies of 1M SrtA-pG and 1M SrtA-pZ are both essentially equivalent.

### DETAILED DESCRIPTION OF THE INVENTION

This disclosure describes near traceless, sortase-mediated bioconjugation methods for generating region-specific ADCs from off-the-shelf human IgG antibodies. As mentioned earlier, Staphylococcus aureus SrtA has been employed for a wide range of protein conjugation applications due its ability to site-specifically attach a molecule modified with a N-terminal oligoglycine peptide to a protein containing a sortase recognition motif, LPXTG (SEQ ID NO: 1). However, this requires genetic modification of IgG to introduce the sortase recognition motif (SRM). To overcome this limitation, this disclosure provides a novel strategy to site-specifically conjugate human IgG antibodies with cargo (e.g., drugs, fluorophores, azide, etc.) via a SrtA-catalyzed isopeptide bond. This isopeptide ligation reaction differs from SrtA's canonical amide ligation reaction in that the isopeptide bond is formed between the carboxyl group of threonine, within the sortase recognition motif, and the ε-amine of a lysine residue. Given that solvent-accessible lysines are naturally present in antibodies, this strategy provides a new method for antibody conjugation that allows site-specific cargo attachment without having to first modify the antibody. In one embodiment, variants of SrtA are fused to an antibody-binding domain, e.g., Protein Z (SrtA-pZ). The Protein Z is used to bring SrtA into close proximity of lysine residues on the IgG heavy chain to improve the efficiency of sortase-mediated isopeptide formation. Upon addition of calcium and a sortase recognition motif (e.g., LPETG (SEQ II) NO: 5)) which can be labeled with a desired cargo, SrtA catalyzes an isopeptide bond between threonine and a defined proximal lysine residue on the Fe fragment, thereby covalently ligating the cargo to the antibody. SrtA-pZ then dissociates from the antibody and is removed from solution after dialysis.

To validate this method, site-specific ADCs were generated using this approach. Monomethyl auristatin E (MMAE) equipped with a LPETG motif via an aminohexanoic acid (Ahx) spacer and a cleavable valine-citrulline (vc) linker were conjugated to Cetuximab, a human IgG1 antibody that targets the epidermal growth factor receptor (EGFR). The structural and functional properties of the ADCs were evaluated to show that the methods described herein can be effectively applied to engineer functional, near traceless, site-specific ADCs from native IgG antibodies.

In one aspect, this disclosure provides facile methods for efficient production of antibody conjugates using antibody-binding domain-transpeptidase fusion proteins. According to one aspect, variants of sortase are used to catalyze the non-canonical isopeptide ligation between peptides possessing a sortase recognition motif and lysines present within the antibody. An antibody-binding domain is fused to the sortase variants to improve the efficiency of isopeptide ligation by bringing the sortase into close proximity to the lysine residues. This approach is referred to as proximity-based sortase-mediated isopeptide ligation (PBS-IL). According to another aspect, the antibody-binding domain-sortase fusion proteins are also able to efficiently label antibodies that have been engineered to possess the sortase recognition motif near the c-terminus of the heavy and/or light chains. In this case, sortase is used to mediate the ligation between the sortase recognition motif and peptides containing an N-terminal glycine. This approach is referred to as proximity-based sortase-mediated protein ligation (PBS-PL). Efficiency of PBS-IL and PBS-PL labeling is significantly improved through the use of sortase variants described herein and the antibody-binding domain, compared with traditional sortase reactions, allowing the use of significantly less peptide in ligation reactions. In each case, the peptide that is conjugated to the antibody can be further labeled with various cargo, including but not limited to, fluorescent dyes, haptens (e.g. biotin), polymers, contrast agents (e.g. gadolinium, radionuclides), chelated metals, therapeutic agents, sensitizers, oligonucleotides, or combinations thereof. The peptides can also be conjugated or fused to additional protein or peptide sequences.

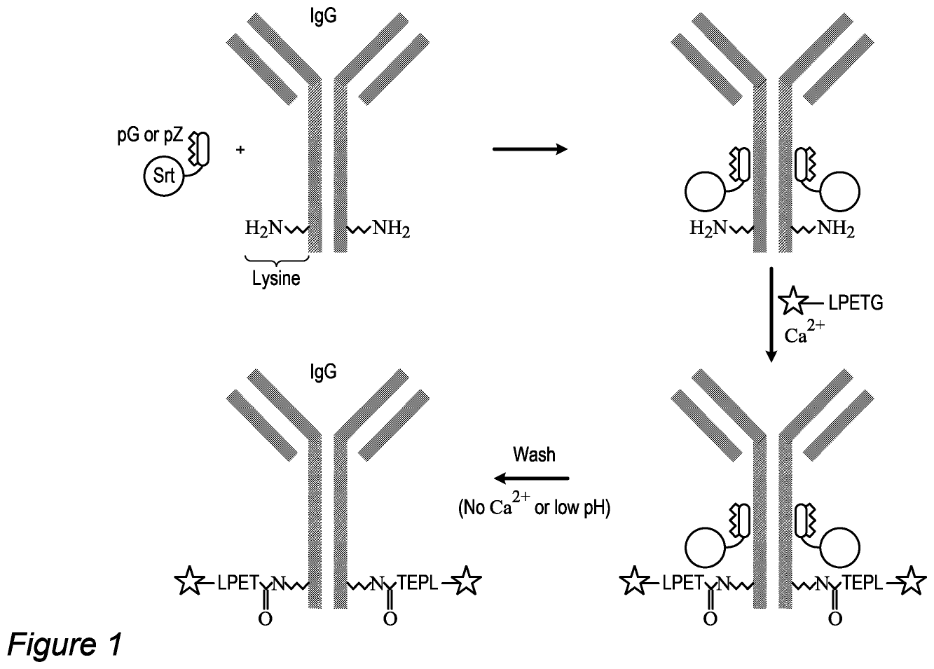

FIG. 1 depicts proximity-based sortase-mediated isopeptide ligation (PBS-IL). The enzyme SrtA is directed to the antibody by an antibody binding domain such as protein G (pG) or protein Z (pZ). For example, SrtA can be directed to the Fc region of an antibody via the fusion protein SrtA-pG or SrtA-pZ. A cargo (e.g., a drug, fluorophore, biotin, PEG, etc., represented by the star) is modified with a SrtA recognition motif (SRM), e.g. LPETG. In the presence of Ca²⁺ and LPETG-modified cargo, SrtA facilitates isopeptide ligation between LPETG-modified cargo and a proximal Fc lysine residue to generate an antibody conjugate. When a calcium-dependent derivative of pZ is used, washing with Ca²⁺-free buffer allows efficient removal of Srt-pZ. Otherwise, an acidic buffer can be used to dissociate pG/pZ from the antibody.

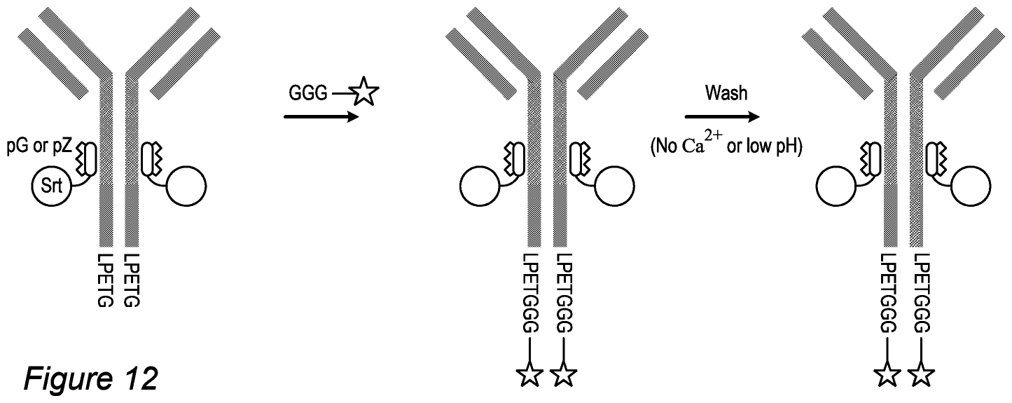

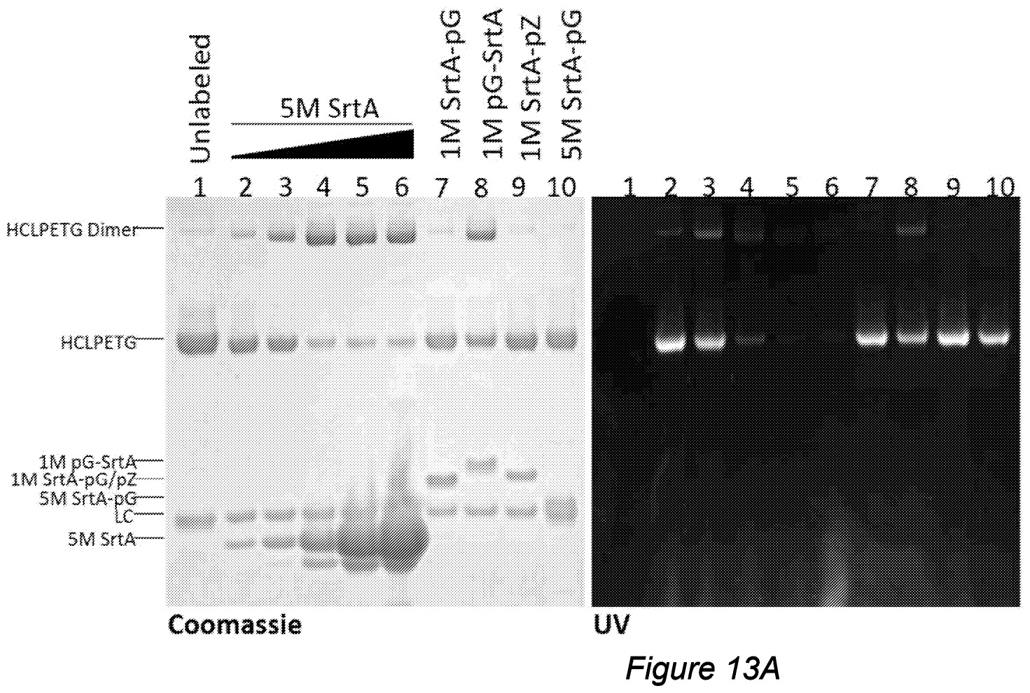

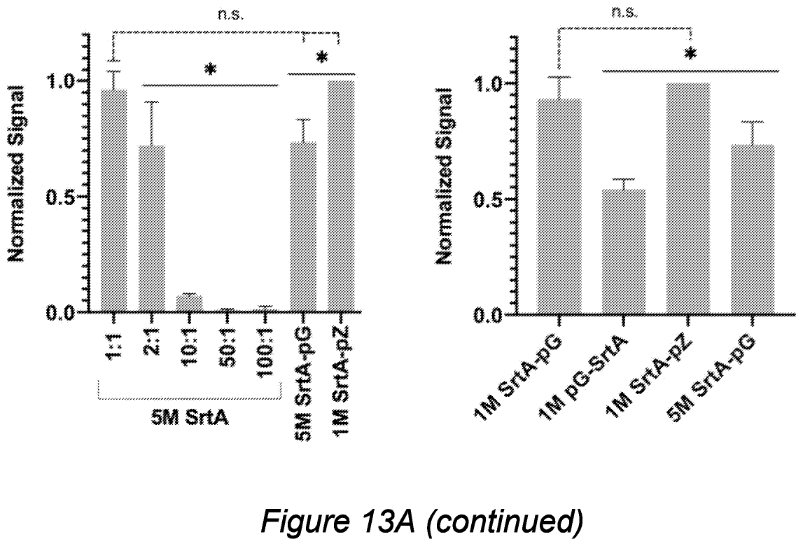

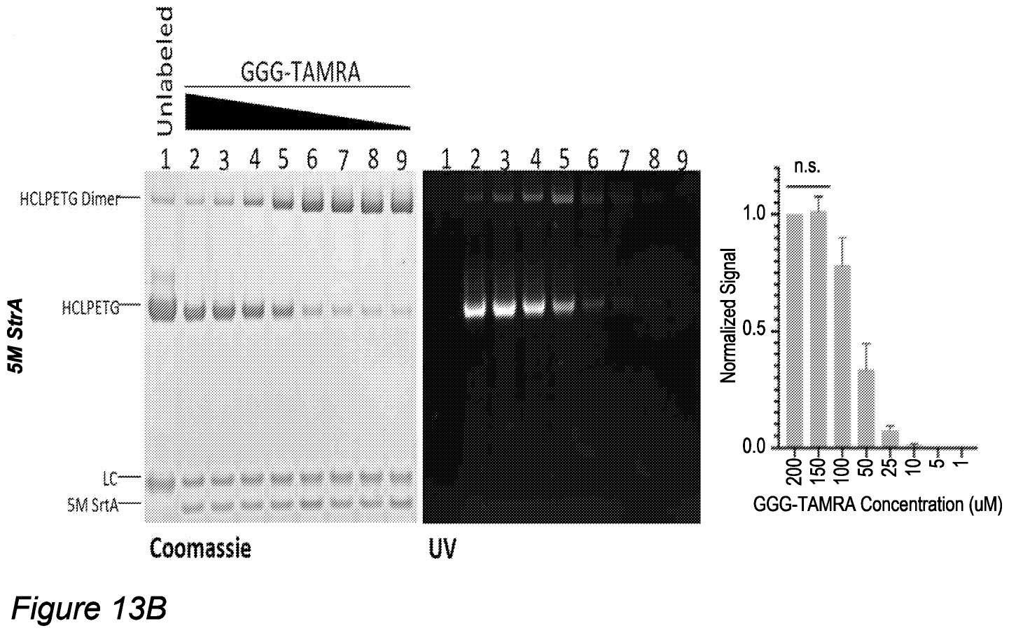

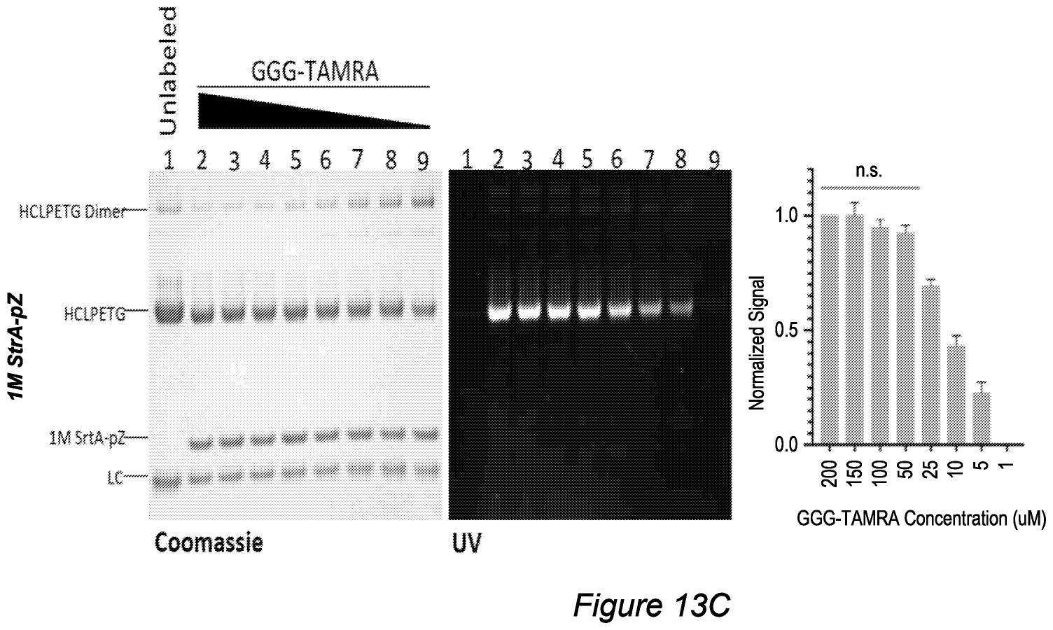

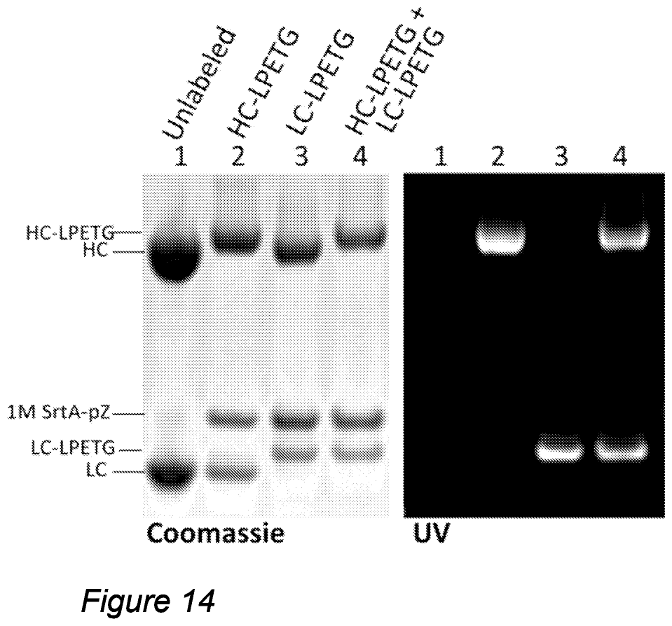

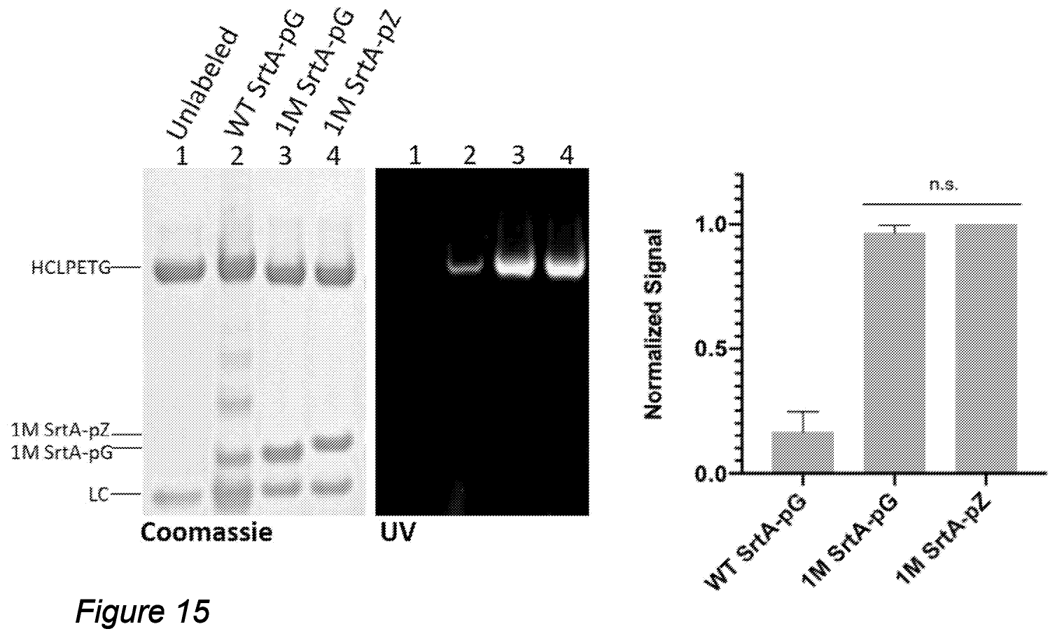

FIG. 12 depicts proximity-based sortase-mediated protein ligation (PBS-PL). A SRM (e.g. LPETG) is fused at the c-terminus of the heavy and/or light chain of IgG. An antibody-binding domain (pG or pZ) is then used to bring sortase (Srt) into close proximity of the SRM to efficiently ligate a drug-labeled peptide (GGG). The pG/pZ-Srt is then removed in a Ca²⁺-free buffer (pZ) or at low pH (pG).

In one aspect, provided herein are compositions for producing an antibody conjugate, the compositions comprise a fusion protein of a sortase and an antibody binding domain that binds to an antibody. The sortases described herein encompass, but are not limited to, sortase A (SrtA), sortase B (SrtB), sortase C (SrtC), sortase D (SrtD), sortase E (SrtE) and sortase F (SrtF). In one embodiment, the sortase is from Gram-positive bacteria. In one embodiment, the sortase is sortase A from Staphylococcus aureus or sortase A from Streptococcus pyogenes. In one embodiment, the sortase is engineered or modified to have unique substrate specificity. In one embodiment, the sortase is engineered or modified to have improved or increased catalytic activity. In one embodiment, the sortase is engineered or modified to be insensitive to calcium.

In some embodiments, fusion proteins described herein comprise sortase A or a variant thereof. In one embodiment, the sortase A comprises one or more of point mutations of T156S, D176E, and D170E. In another embodiment, the sortase A comprises a point mutation of N127K. In one embodiment, the antibody binding domain in fusion proteins described herein can be protein G, protein A, a protein G variant, a protein A variant, or a subdomain of protein G or protein A. In one embodiment, the antibody binding domain is selected from a Protein G HTB1 domain, a Protein Z domain, a Protein A, a Protein G, a Protein L, a Protein LG, a Protein LA, a Protein A/G, or an Fe-binding peptide, such as Fc-III, Fc-III-4C, APAR, PAM, FcBP-2, RRGW, KHRFNKD, or a functional sub-domains thereof. In some embodiments, the antibody binding domain binds to an immunoglobulin G (IgG), an immunoglobulin M (IgM), an immunoglobulin D (IgD), an immunoglobulin E (IgE), or an immunoglobulin A (IgA). In human, the IgG can be IgG1, IgG2, IgG3, or IgG4.

In some embodiments, provided herein are methods of producing an antibody conjugate, comprising the steps of: (i) contacting an antibody with a fusion protein comprising a sortase and an antibody binding domain, wherein the antibody binding domain binds to the antibody; and (ii) linking a peptide that comprises a sortase recognition motif to the antibody via a ligation mediated by the sortase, wherein a isopeptide bond is formed between the peptide and a lysine on the antibody, thereby forming an antibody conjugate comprising the peptide. In one embodiment, the sortase is sortase A or a variant thereof. In one embodiment, the sortase A comprises one or more of point mutations of T156S, D176E, and D170E. In another embodiment, the sortase A comprises a point mutation of N127K. In one embodiment, the antibody binding domain is protein G, protein A, a protein G variant, a protein A variant, or a subdomain of protein G or protein A. In one embodiment, the antibody binding domain is selected from a Protein G HTB1 domain, a Protein Z domain, a Protein A, a Protein G, a Protein L, a Protein LG, a Protein LA, a Protein A/G, or an Fc-binding peptide, such as Fc-III, Fc-III-4C, APAR, PAM, FcBP-2, RRGW, KHRFNKD, or a functional sub-domains thereof. In some embodiments, the antibody binding domain binds to an immunoglobulin G (IgG), an immunoglobulin M (IgM), an immunoglobulin D (IgD), an immunoglobulin E (IgE), or an immunoglobulin A (IgA). In one embodiment, the peptide further comprises a fluorescent dye, a hapten, a polymer, a contrast agent, a radionuclide, a chelated metal, a therapeutic agent, a sensitizer, an oligonucleotide, or combinations thereof.

In one embodiment, the sortase recognition sequence can be one of LPXTG (SEQ ID NO: 1), LPKTG (SEQ ID NO: 2), LPATG (SEQ ID NO: 3), LPNTG (SEQ ID NO: 4), LPETG (SEQ ID NO: 5), LPXAG (SEQ ID NO: 6), LPNAG (SEQ ID NO: 7), LPXTA (SEQ ID NO: 8), LPNTA (SEQ ID NO: 9), LGXTG (SEQ ID NO: 10), LGATG (SEQ ID NO: 11), IPXTG (SEQ ID NO: 12), IPNTG (SEQ ID NO: 13), IPETG (SEQ ID NO: 14), NPQTN (SEQ ID NO: 15), LAXTG (SEQ ID NO: 16), LPXSG (SEQ ID NO: 17), LSETG (SEQ ID NO: 18), LPXCG (SEQ ID NO: 19), LPXAG (SEQ ID NO: 20), and XPETG (SEQ ID NO: 21).

In one embodiment, provided herein are methods of producing an antibody conjugate, comprising the steps of: (i) contacting an antibody with a fusion protein comprising a sortase and an antibody binding domain, wherein the antibody binding domain binds to the antibody, and the antibody comprises a sortase recognition motif near the C-terminus of one or both of its heavy chain and light chain; and (ii) linking a peptide comprising a N-terminus glycine to the antibody via a ligation mediated by the sortase, wherein a peptide bond is formed between the peptide and the sortase recognition motif on the antibody, thereby forming an antibody conjugate comprising the peptide. In one embodiment, the sortase is sortase A or a variant thereof. In one embodiment, the sortase A comprises one or more of point mutations of T156S, D176E, and D170E. In another embodiment, the sortase A comprises a point mutation of N127K. In one embodiment, the sortase recognition motif has the sequence of one of SEQ ID NOs: 1-21 as disclosed above. In one embodiment, the antibody binding domain is protein G, protein A, a protein G variant, a protein A variant, or a subdomain of protein G or protein A. In one embodiment, the antibody binding domain is selected from a Protein G HTB1 domain, a Protein Z domain, a Protein A, a Protein G, a Protein L, a Protein LG, a Protein LA, a Protein A/G, or an Fc-binding peptide, such as Fc-III, Fc-III-4C, APAR, PAM, FcBP-2, RRGW, KHRFNKD, or a functional sub-domains thereof. In some embodiments, the antibody binding domain binds to an immunoglobulin G (IgG), an immunoglobulin M (IgM), an immunoglobulin D (IgD), an immunoglobulin E (IgE), or an immunoglobulin A (IgA). In one embodiment, the peptide further comprises a fluorescent dye, a hapten, a polymer, a contrast agent, a radionuclide, a chelated metal, a therapeutic agent, a sensitizer, an oligonucleotide, or combinations thereof.

In some embodiments, the N-terminal glycine comprises a single glycine. In some embodiments, the N-terminal glycine comprises a plurality of N-terminal glycines or an N-terminal polyglycine, such as an N-terminal triglycine. In some embodiments, the glycine, polyglycine, or peptide/protein (including enzymes) with an N-terminal glycine further comprises a functional group or label. In some embodiments, the glycine, polyglycine, or peptide/protein with an N-terminal glycine is fused or linked to a protein, an enzyme, a drug molecule, an imaging agent, a metal chelate, a polyethylene glycol, a click chemistry group, an alkyne, an azide, a hapten, a biotin, a photocrosslinker, an oligonucleotide, a small molecule, a nanoparticle, or an antibody binding domain.

In one embodiment, provided herein are isolated polynucleotides encoding a fusion protein comprising a sortase and an antibody binding domain that binds to an antibody. In one embodiment, the sortase is sortase A or a variant thereof. In one embodiment, the sortase A comprises one or more of point mutations of T156S, D176E, and D170E. In another embodiment, the sortase A comprises a point mutation of N127K. In one embodiment, the antibody binding domain is protein G, protein A, a protein G variant, a protein A variant, or a subdomain of protein G or protein A. In one embodiment, the antibody binding domain is selected from a Protein G HTB1 domain, a Protein Z domain, a Protein A, a Protein G, a Protein L, a Protein LG, a Protein LA, a Protein A/G, or an Fc-binding peptide, such as Fe-Ill, Fc-Ill-4C, APAR, PAM, FcBP-2, RRGW, KHRFNKD, or a functional sub-domains thereof. In another embodiment, provided herein are vectors comprising the isolated polynucleotide discussed above. In yet another embodiment, provided herein are cells comprising the vector discussed above.

As used herein, “antibody” refers to a protein consisting of one or more polypeptides substantially encoded by all or part of the recognized immunoglobulin genes. The recognized immunoglobulin genes, for example in humans, include the kappa (κ) lambda (λ) and heavy chain genetic loci, which together comprise the myriad variable region genes, and the constant region genes mu (μ), delta (s), gamma (γ), sigma (σ) and alpha (α) which encode the IgM, IgD, IgG, IgE, and IgA isotypes or classes, respectively. The term “antibody” is meant to include full-length antibodies, and may refer to a natural antibody from any organism, an engineered antibody, or an antibody generated recombinantly for experimental, therapeutic, or other purposes. By “full length antibody” herein is meant the structure that constitutes the natural biological form of an antibody, including variable and constant regions. As used herein, the term “antibody” comprises monoclonal and polyclonal antibodies. Antibodies can be antagonists, agonists, neutralizing, inhibitory, or stimulatory.

As used herein, “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3.

The amino acid sequence of the wild-type Staphylococcus aureus Sortase A (SrtA) is MQAKPQIPKDKSKVAGYIEIPDADIKEPVYPGPATPEQLNRGVSFAEENESLDDQNISIAG HTFIDRPNYQFTNLKAAKKGSMVYFKVGNETRKYKMTSIRDVKPTDVEVLDEQKGKD KQLTLITCDDYNEKTGVWEKRKIFVATEVK (SEQ ID NO: 24). A Sortase A amino acid sequence may also include homologous, variant, and functional fragment sequences of a Sortase A. One of ordinary skill in the art would readily use techniques generally known in the art to generate a variant of Sortase A. In some embodiments, the Sortase A amino acid sequence may include an amino acid sequence which is 60, 65, 70, 75, 80, 85, 90, 95, 96, 97, 98 or 99% identical to the sequence set forth in SEQ ID NO: 24.

In one aspect, provided herein are sortase variants that catalyze an isopeptide ligation reaction. In some embodiments, the sortase variant is a sortase A variant. In one embodiment, the sortase A variant comprises one or more of point mutations of T156S, D176E, and D170E. In another embodiment, the sortase A variant comprises a point mutation of N127K. In some embodiments, the Sortase A variant has the amino acid sequence set forth in SEQ ID NO: 25. In some embodiments, the Sortase A variant has the amino acid sequence set forth in SEQ ID NO: 26. In some embodiments, the amino acid sequence of the sortase A variant is 60, 65, 70, 75, 80, 85, 90, 95, 96, 97, 98 or 99% identical to one of the sequences set forth in SEQ ID NOs: 24-26.

In one embodiment, provided herein are isolated polynucleotides encoding a sortase described herein. In one embodiment, the sortase is a sortase A or a variant thereof. In one embodiment, the sortase A comprises one or more of point mutations of T156S, D176E, and D170E. In another embodiment, the sortase A comprises a point mutation of N127K. In another embodiment, provided herein are vectors comprising the isolated polynucleotide encoding a sortase described herein. In yet another embodiment, provided herein are cells comprising the vector comprising the isolated polynucleotide encoding a sortase described herein.

As used herein, “Protein Z” refers to the Z domain which is an analog of the B domain of Staphylococcal aureus Protein A. The amino acid sequence of wild-type Protein Z is: VDNKFNKEQQNAFYEILHLPNLNEEQRNAFIQSLKDDPSQSANLLAEAKKLNDAQAPK MRM (SEQ ID NO: 22). A Protein Z amino acid sequence may also include homologous, variant, and fragment sequences having Z domain function. One of ordinary skill in the art would readily use techniques generally known in the art to generate a variant of protein Z. In some embodiments, the Protein Z amino acid sequence may include an amino acid sequence which is 60, 65, 70, 75, 80, 85, 90, 95, 96, 97, 98 or 99% identical to the sequence set forth in SEQ ID NO: 22.

As used herein, “Protein G” refers to a B1 domain of Streptococcal Protein G. In one embodiment, the Protein G is a hypothermophilic variant of a B1 domain of Streptococcal Protein G. In one embodiment, the amino acid sequence of Protein G is: MTFKLIINGKTLKGEITIEAVDAAEAEKIFKQYANDYGIDGEWTYDDATKTFTVTE (SEQ ID NO: 23). The Protein G amino acid sequence may also include homologous, variant, and fragment sequences having B1 domain function. One of ordinary skill in the art would readily use techniques generally known in the art to generate a variant of protein G. In some embodiments, the Protein G amino acid sequence may include an amino acid sequence which is 60, 65, 70, 75, 80, 85, 90, 95, 96, 97, 98 or 99% identical to the sequence set forth in SEQ ID NO: 23.

As used herein, the terms “binds” or “binding” or grammatical equivalents, refer to compositions having affinity for each other. “Specific binding” is where the binding is selective between two molecules. An example of specific binding is that which occurs between an antibody and an antigen. Typically, specific binding can be distinguished from non-specific when the dissociation constant (K_{D}) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In one embodiment, a combination of proteins or biologically active agents such as a cytokine, an enzyme, a chemokine, a radioisotope, an enzymatically active toxin, or a chemotherapeutic agent can be applied to the compositions and methods provided herein.

In one embodiment, a variety of radioactive isotopes are available to produce radio-conjugate antibodies and can be of use in the methods and compositions provided herein. Examples include, but are not limited to, At²¹¹, Cu⁶⁴, I¹³¹, I¹²⁵, Y⁹⁰, Re¹⁸⁶, Re¹⁸⁸, Sm¹⁵³, Bi²¹², P³², and radioactive isotopes of Lu.

In another embodiment, enzymatically active toxin or fragments thereof that can be used in the compositions and methods provided herein include, but are not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In one embodiment, a chemotherapeutic or other cytotoxic agent may be conjugated to an antibody or immunoglobulin according to the methods provided herein as an active drug or as a prodrug. A “prodrug” refers to a precursor or derivative form of a pharmaceutically active substance that is less cytotoxic to tumor cells compared to the parent drug and is capable of being enzymatically activated or converted into the more active parent form. See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985. The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

In one embodiment, a variety of other therapeutic agents may find use for administration with the antibodies and conjugates of the compositions and methods provided herein. In one embodiment, the conjugate comprising an antibody is administered with an anti-angiogenic agent. As used herein, the term “anti-angiogenic agent” refers to a compound that blocks, or interferes to some degree, the development of blood vessels. The anti-angiogenic factor may, for instance, be a small molecule or a protein, for example an antibody, Fc fusion, or cytokine, that binds to a growth factor or growth factor receptor involved in promoting angiogenesis. In an alternate embodiment, the conjugate is administered with a therapeutic agent that induces or enhances adaptive immune response. In an alternate embodiment, the conjugate is administered with a tyrosine kinase inhibitor. The term “tyrosine kinase inhibitor” refers to a molecule that inhibits to some extent tyrosine kinase activity of a tyrosine kinase as known in the art.

In one embodiment, the conjugates provided herein may be used for various therapeutic purposes. In one embodiment, the conjugates are administered to a subject to treat an antibody-related disorder. In another embodiment, the conjugate proteins are administered to a subject to treat a tumor or a cancer tumor. A “subject” for the purposes of the compositions and methods provided herein includes humans and other animals, preferably mammals and most preferably humans. Thus the conjugates provided herein have both human therapy and veterinary applications. In another embodiment, the subject is a mammal, and in yet another embodiment the subject is human. By “condition” or “disease” herein are meant a disorder that may be ameliorated by the administration of a pharmaceutical composition comprising the conjugate of the compositions and methods provided herein. Antibody related disorders include but are not limited to autoimmune diseases, immunological diseases, infectious diseases, inflammatory diseases, neurological diseases, and oncological and neoplastic diseases including cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description herein, steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Example 1

### Generation of Improved Sortase A Mutants for Isopeptide Ligation

### Materials and Methods

Random Mutagenesis by Error-Prone PCR. Error-prone PCR (EP-PCR) was performed to introduce random point mutations into the wild-type SrtA gene using the GeneMorph II Random Mutagenesis Kit (Agilent). Approximately 2 ng of the pSTEPL vector was mutagenized to achieve a high mutation frequency. Forward and reverse primers were designed to incorporate NdeI and BamHI sites respectively. PCR products were then separated by gel electrophoresis, and the mutagenized template DNA was recovered using a gel-extraction kit (Qiagen). The template DNA was digested at the NdeI and BamHI sites, purified via a PCR purification kit (Qiagen), and then ligated into pRSET-A (Invitrogen), which contained (GGS)₂ linker and a C-terminal Protein G. The mutated SrtA was cloned upstream of the (GGS)₂ linker and Protein G to generate a plasmid encoding the SrtA-Protein G (SrtA-pG) fusion protein. The cloned vector was transformed into T7 Express Competent E. coli cells (New England BioLabs), which were spread onto Luria Broth (LB) agar plates containing ampicillin (100 μg/mL) and grown overnight at 37° C. Around 400 colonies were handpicked and cultured in 96 deep-well plates containing 1 mL of auto induction medium (Formedium), supplemented with ampicillin and glycerol, at 25° C. for 48 hours. Subsequently, glycerol stocks were prepared for each clone by transferring 75 μL of bacterial cultures to 75 μL of 50% glycerol, which were stored at −80° C. for future library expression. Remaining cultures from 96-well deep plates were collected by centrifugation and then lysed using 200 μL of lysis buffer (1% octylthioglucoside) per well. Cell lysates were clarified via centrifugation (4000 rpm, 5 min).

Screening of Mutant Libraries. SrtA-pG mutants were selected using a screening assay that measured the degree of labeling on IgG antibodies by each mutant. At separate times, SrtA-pG was evolved against two different IgG1 antibodies (Trastuzumab and Cetuximab) to identify universal mutations to improve antibody labeling. Using transparent 96-well half-area plates (Corning), 100 ng of either Trastuzumab or Cetuximab in phosphate buffered saline (PBS) was coated per well overnight at 4 C. In parallel, corresponding plates were also coated with PBS only as a negative control. The following day, plates were decanted and blocked with Blocking Buffer (1×PBS, 0.1% Tween 20, 0.3% bovine serum albumin (BSA)) for 1 hour at room temperature while shaking. After blocking and decanting, 20 μL of the clarified lysate for each clone was added to the antibody-coated plate and the PBS-coated plate in parallel, followed by 30 μL of a 10 μM Biotinvc-Ahx-LPETG and 167 μM CaCl₂ mixture per well. The plate was then incubated for 1 hour at 37° C., after which the plate was washed three times with PBST (1×PBS, 0.05% Tween 20). Next, streptavidin-HRP (Thermo Scientific) diluted 1/8000 in Blocking Buffer was added to plates, incubating for 45 min at room temperature while shaking. Wash steps were repeated, and plates were developed with TMB substrate solution (Pierce) for 5 min before the adding 2 M sulfuric acid to stop the reaction. Absorbance signal was detected at 450 nm using a Tecan Infinite M200 plate reader. Assay background was normalized by subtracting the negative control signal from the antibody-coated plate signal for each corresponding clone.

Mutant Characterization and Selection. Top 3-4 clones with the highest normalized absorbance values from each plate were sequenced. From glycerol stocks, SrtA-pG clones with novel point mutations were then expressed in 300 mL of auto induction medium with ampicillin and glycerol at 37° C. overnight. Cells were harvested via centrifugation (5000 rpm, 10 min) and then lysed with lysis buffer (1% octylthioglucoside) for 30 min while rotating. Cell lysates were aliquoted into 2 mL tubes and clarified using a benchtop centrifuge (14000 rpm, 25 min). Clarified lysates were incubated with HisPur Cobalt Resin (Thermo Scientific) in a column while rotating for 30 min. Bound resin was washed with 1×PBS and then eluted with 200 mM imidazole. Protein elutions were dialyzed in 1 L of PBS overnight at 4° C. and then run on a SDS-PAGE gel to check protein yield. Once proteins were purified, the labeling efficiencies of the SrtA-pG clones were then compared against that of WT SrtA-pG. Labeling reactions in 10 μl volumes were prepared as follows: 2 μg Cetuximab or Trastuzumab, 200 μM TAMRAvc-Ahx-LPETG (referred to as TAMRA-LPETG hereafter), 50 μM CaCl₂, and 1.25 μM SrtA-pG. After incubating at 37° C. in the dark overnight, labeling reactions were run on a SDS-PAGE gel, which was imaged in UV and white light and then analyzed via ImageJ. Labeling efficiency on the heavy chain was measured by the UV/SDS ratio of the fluorescent band intensity to the protein band intensity. The SrtA-pG clone with the greatest labeling activity compared against that of WT SrtA-pG was selected and served as the template for the next round of mutagenesis. A total of 3 rounds were conducted to evolve SrtA-pG against Cetuximab to produce a tri-mutant SrtA-pG (3M SrtA-pG). Meanwhile, for Trastuzumab, one round was sufficient to evolve SrtA-pG with superior labeling efficiency to yield a single mutant SrtA-pG (1M SrtA-pG). 1M and 3M SrtA-pG were later re-cloned to replace Protein G with Protein Z to generate 1M and 3M SrtA-pZ, which improved solubility and elution conditions.

Measurement of Labeling Efficiency. The labeling efficiencies of the evolved SrtA-pG/pZ mutants were compared to those of well-studied SrtA variants, wild-type SrtA (WT SrtA) and pentamutant SrtA (5M SrtA). In addition, WT and 5M SrtA fused to Protein G, as well as a 1M SrtA-pG in the opposite orientation (1M pG-SrtA), were also evaluated. All SrtA variants were cloned in pRSET-A (Invitrogen) and expressed in T7 Express Competent E. coli cells (New England BioLabs). Bacterial starter cultures for each protein were grown in 2 mL of LB broth supplemented with 100 μg/mL ampicillin were grown at 37° C. overnight. The next day, 1 mL of starter culture was transferred to 300 mL of auto induction medium (Formedium) with ampicillin and glycerol at 37° C. overnight. After purification with HisPur Cobalt Resin (Thermo Scientific), proteins were dialyzed in 1 L of PBS overnight at 4° C. Labeling reactions for each SrtA variant were prepared with the following conditions: 2 μg Cetuximab, 200 μM TAMRA-LPETG, 50 μM CaCl₂, and 1.25 μM SrtA variant in 10 mM Tris-HCl buffer. Each reaction was incubated at 37° C. overnight in the dark and then run on a SDS-PAGE gel. The gel was imaged in UV and white light and then analyzed on ImageJ. Labeling efficiency was measured by calculating the UV/SDS ratio of the fluorescent heavy chain band intensity to the heavy chain protein band intensity. All UV/SDS ratios were normalized by UV/SDS ratio of either 1M SrtA-pG or 1M SrtA-pZ to compare the labeling efficiencies of other SrtA variants relative to 1M SrtA-pG or 1M SrtA-pZ. TAMRA-LPETG and 1M SrtA-pZ titration experiments to optimize labeling conditions for Cetuximab were also done by measuring the labeling efficiency of each set of reaction conditions. In these studies, the UV/SDS ratio for each condition was normalized by the maximum UV/SDS ratio in that experiment. All labeling experiments were repeated three times.

TABLE 1
Sortase A Sequences

| Name | Amino Acid Sequence |
|---|---|
| SrtA | MQAKPQIPKDKSKVAGYIEIPDADIKEPVYPGPATPEQLNRGVSF |
| WT | ÅEENESLDDQNISIAGHTFIDRPNYQFTNLKAAKKGSMVYFKVGN |
| | ETRKYKMTSIRDVKPTDVEVLDEQKGKDKQLTLITCDDYNEKTGV |
| | WEKRKIFVATEVK (SEQ ID NO: 24) |
| SrtA | MQAKPQIPKDKSKVAGYIEIPDADIKEPVYPGPATPEQLNRGVSF |
| 1M | AEENESLDDQNISIAGHTFIDRPKYQFTNLKAAKKGSMVYFKVGN |
| | ETRKYKMTSIRDVKPTDVEVLDEQKGKDKQLTLITCDDYNEKTGV |
| | WEKRKIFVATEVK (SEQ ID NO: 25) |
| SrtA | MQAKPQIPKDKSKVAGYIEIPDADIKEPVYPGPATPEQLNRGVSF |
| 3M | AEENESLDDQNISIAGHTFIDRPNYQFTNLKAAKKGSMVYFKVGN |
| | ETRKYKMSSIRDVKPTDVEVLEEQKGKEKQLTLITCDDYNEKTGV |
| | WEKRKIFVATEVK (SEQ ID NO: 26) |
| SrtA | MQAKPQIPKDKSKVAGYIEIPDADIKEPVYPGPATREQLNRGVSF |
| 5M | AEENESLDDQNISIAGHTFIDRPNYQFTNLKAAKKGSMVYFKVGN |
| | ETRKYKMTSIRNVKPTAVEVLDEQKGKDKQLTLITCDDYNEETGV |
| | WETRKIFVATEVK (SEQ ID NO: 27) |

Synthesis of Site-Specific Cetuximab-Ahx-vcTAMRA and Cetuximab-Ahx-vcMMAE Conjugates. Cetuximab was conjugated with TAMRA or Monomethyl auristatin E (MMAE) using 1M SrtA-pZ, which demonstrated superior labeling efficiency, to synthesize the Cetuximab-Ahx-vcTAMRA and Cetuximab-Ahx-vcMMAE conjugates. First, a 500 μL reaction was prepared by mixing the following components in 10 mM Tris-HCl buffer: 500 μg of Cetuximab, 200 μM TAMRAvc-Ahx-LPETG or MMAEvc-Ahx-LPETG, 500 μM CaCl₂), and 1.25 μM 1M SrtA-pZ. The reaction was incubated at 37° C. in the dark overnight. The next day, the reaction was dialyzed in 1 L of sterile PBS for 4 hours at 4° C. to remove excess reaction components. After, the dialysis buffer was replaced, and the sample was dialyzed for another 24-48 hours. Following dialysis, the sample was collected and then run on a SDS-PAGE gel to check yield. The concentration was quantified by ImageJ and Nanodrop.

Quantification of Drug-to-Antibody Ratio. The average drug-to-antibody ratio (DAR) was determined by UV/Vis spectroscopy. Quantification of the average DAR was performed for Cetuximab-Ahx-vcTAMRA, assuming that the DARs of Cetuximab-Ahx-vcTAMRA and Cetuximab-Ahx-vcMMAE are similar. The protocol used here for measuring the average DAR via UV/Vis Spectroscopy has been detailed previously. First, using NanoDrop, known concentrations of TAMRA-LPETG (16 μM) and Cetuximab (13.33 μM) was measured for absorbance at 280 and 555 nm (A₂₈₀ and A₅₅₅), which correspond to the absorption maxima for protein and TAMRA, respectively. The A₂₈₀ and A₅₅₅ values for Cetuximab-Ahx-vcTAMRA were also measured. Using the Beer-Lambert law with a path length of 0.1 cm, the extinction coefficients of TAMRA at 280 and 555 nm were determined from the TAMRA-LPETG concentration and absorbance measurements. Using the same method, the extinction coefficients for Cetuximab at 280 and 555 nm were calculated. Since Cetuximab-Ahx-vcTAMRA has some A₂₈₀ contribution from the peptide linker, the A₂₈₀ measurement was corrected so that the A₂₈₀ of Cetuximab-Ahx-vcTAMRA is entirely from the protein contribution of Cetuximab. This was done by multiplying the A₅₅₅ of Cetuximab-Ahx-vcTAMRA with a correction factor (A₂₈₀ of TAMRA-LPETG divided by A₅₅₅ of TAMRA-LPETG) to calculate the A₂₈₀ contribution from the peptide linker. This background A₂₈₀ value was subtracted from the A₂₈₀ of Cetuximab-Ahx-vcTAMRA. From the A₂₈₀ and A₅₅₅ measurements and the calculated extinction coefficients, the individual concentrations of Cetuximab and TAMRA in the Cetuximab-Ahx-vcTAMRA sample were determined using the Beer-Lambert law for a multi-component system. The average DAR was calculated by dividing the Cetuximab concentration by the TAMRA concentration.

Fluorescence Microscopy. MDA-MB 468 cells were passaged in Dulbecco's Modified Eagle Medium (DMEM) supplemented by 10% fetal bovine serum (FBS) and 5% penicillin-streptomycin. A total of 125,000 cells were seeded per well on a 48-well tissue culture plate (Falcon). After 24 hours, cells were fixed for 15 min using neutral buffered formalin solution (Sigma-Aldrich) and then washed three times with PBST (1×PBS, 0.05% Tween 20). Fixed cells were then resuspended in PBST and blocked with 10% Normal Goat Serum (Life Technologies) for 15 min. After blocking, wash steps were repeated. Fixed cells were then incubated with either 60 nM of Cetuximab-Ahx-vcTAMRA or 60 nM of Cetuximab for 1 hour at room temperature while shaking in the dark. After, cells were washed again to remove excess Cetuximab-Ahx-vcTAMRA or Cetuximab and then resuspended in PBST. Imaging was performed on an Olympus IX81 and then analyzed on ImageJ.

Fixed Cell-Surface Ligand Binding Assay. MDA-MB 468 cells were seeded in black, transparent-bottom plates (Corning) at a density of 10,000 cells per well. Once 70-80% confluency was reached, cells were fixed with neutral buffered formalin solution (Sigma-Aldrich) for 15 min and then washed three times with PBST (1×PBS, 0.05% Tween 20). Cells were blocked with 10% Normal Goat Serum (Life Technologies) for 15 min and washed three times again with PBST. For the binding step, Cetuximab-Ahx-vcTAMRA was added to cells in triplicates at a starting concentration of 30 nM, which was serially diluted three-fold to 0.000508 nM in Blocking Buffer (1×PBS, 0.1% Tween 20, 0.3% BSA). Triplicate negative control wells with only Blocking Buffer were also included. The same triplicate dilutions for Cetuximab alone were also added to adjacent wells in parallel. After binding for 1 hour covered while shaking, wash steps were repeated. Next, Goat Anti-Human IgG Fc Secondary Antibody, PE (eBiosciences) diluted 1/1000 in Blocking Buffer was added to cells and incubated for 1 hour covered while shaking. Wash steps were repeated, and fixed cells were resuspended in PBS. Fluorescent signal was measured at 544/585 nm on a Tecan M200 Infinite plate reader. Raw signals were normalized by subtracting the background (negative controls) and then dividing by the maximum signal for each condition. Normalized data was analyzed using GraphPad Prism to calculate the binding affinity constants (K_{D}).

Neonatal Fc Receptor Binding Assay. Half of a transparent 96-well half-area plate (Corning) was coated with 0.5 μg of Cetuximab-Ahx-vcMMAE diluted in PBS per well at 4° C. overnight. In parallel, the other half of the plate was coated with 0.5 μg of Cetuximab alone diluted in PBS per well. The next day, the plate was washed four times with Wash Buffer (1×Tris-buffered saline (TBS), 0.05% Tween 20, pH 7.4). Plates were then blocked with Blocking Buffer (2% BSA in Wash Buffer 1, pH 7.4) at 37° C. for 1.5 hours. After, plates were decanted, and wash steps were repeated with Wash Buffer 1. Biotinylated Human Neonatal Fc Receptor (FcRn)+B2M Heterodimer (Acro Biosystems) diluted in Dilution Buffer (0.5% BSA in Wash Buffer 2, pH 6.0) was then added to Cetuximab-Ahx-vcMMAE- and Cetuximab-coated wells in duplicate. A starting concentration of 0.625 μg/mL was added, which was serially diluted two-fold to 0.00061 μg/mL. Duplicate negative control wells with Dilution Buffer only were also included for each condition. The plate was incubated at 37° C. for 1 hour and then wash steps were repeated using Wash Buffer 2 (1×TBS, 0.05% Tween 20, pH 6.0). For detection, streptavidin-HRP (Thermo Fisher) diluted 1/10000 in Dilution Buffer was added to the plate, which was incubated at 37° C. for 1 hour. Wash steps were repeated with Wash Buffer 2. Next, the plate was developed with TMB substrate solution (Pierce) at 37° C. for 20 min in the dark. 2 M sulfuric acid was added to stop the TMB reaction, and the plate was measured for absorbance at 450 nm with a Tecan M200 Infinite plate reader. Data were analyzed using GraphPad Prism.

Fc-gamma Receptor/Binding Assay. A transparent 96-well half-area plate (Corning) was coated using the same protocol described for the FcRn binding assay. The next day, the plate was washed four times with Wash Buffer 1 (1×TBS, 0.05% Tween 20, pH 7.4) and then blocked with Blocking Buffer (2% BSA in Wash Buffer 1, pH 7.4) at 4° C. for 1.5 hours. Wash steps were repeated. Biotinylated Human CD64/Fc-gamma Receptor 1 (FcγRI) diluted in Dilution Buffer (0.5% BSA in Wash Buffer 1) was added to Cetuximab-Ahx-vcMMAE- and Cetuximab-coated wells in duplicate at a starting concentration of 100 ng/mL, which was serially diluted two-fold to 0.78 ng/mL. Duplicate negative control wells with Dilution Buffer only were also included for each condition. The plate was incubated for 1 hour at room temperature while shaking. After, wash steps were repeated, and the plate was incubated with streptavidin-HRP (Thermo Fisher) diluted 1/10000 in Dilution Buffer for 1 hour at room temperature while shaking. Wash steps were repeated again. The plate was then developed in the dark using TMB substrate solution (Pierce). After 10 min, the TMB reaction was stopped using 2 M sulfuric acid, and the plate was measured for absorbance at 450 nm using a Tecan M200 Infinite plate reader. Data were analyzed using GraphPad Prism.

Cytolysis Assay. Cytolysis assays were performed using the xCELLigence Real-Time Cell Analyzer (RTCA) system (ACEA Biosciences), which measures the electrical impedance, or cell index, from adherent cells to quantify cell proliferation. Both MDA-MB 468 and A431 cell lines, which overexpress epidermal growth factor receptor (EGFR), were assayed. All cell lines were cultured in DMEM supplemented with 10% FBS and 5% penicillin-streptomycin. First, a 96-well Electronic Microtiter Plate (ACEA Biosciences) was scanned with media only to get a background reading using the xCELLigence RTCA instrument. Cells were then seeded at a density of 5000 cells per well and left at room temperature for 30 min to allow the cells to settle before placing the plate back into the instrument. The next day, cells were treated with Cetuximab-Ahx-vcMMAE, Cetuximab mixed with free MMAE, or Cetuximab alone in duplicate. For Cetuximab-Ahx-vcMMAE and Cetuximab conditions, the starting concentration added was 125 nM, which was serially diluted three-fold to 0.02 nM. Cetuximab mixed with free MMAE was added at a 1:2 molar ratio, such that starting concentrations of Cetuximab and free MMAE were 125 μM and 250 μM, respectively. This condition was serially diluted three-fold in the same way. Duplicate untreated wells with PBS only were also included for each condition and were designated as 100% viability controls. After 96 hours, cell index data was collected. Data normalization was performed to calculate the degree of cytolysis (% cytolysis) in treated cells relative to the untreated conditions. This value was determined by calculating the amount of cell killing (difference between the cell indexes of treated and untreated cells) and then dividing by the cell index of untreated cells (no cytolysis). The data was analyzed using GraphPad Prism to calculate the EC₅₀, or the drug concentration that yields a half-maximal cytolytic response.

Statistical Analysis. For all labeling experiments, one-way analysis of variance (ANOVA) was used to determine statistical significance, followed by Tukey's multiple comparison test. P values were considered significant for p≤0.05. GraphPad Prism was used to perform all statistical analyses.

### Results

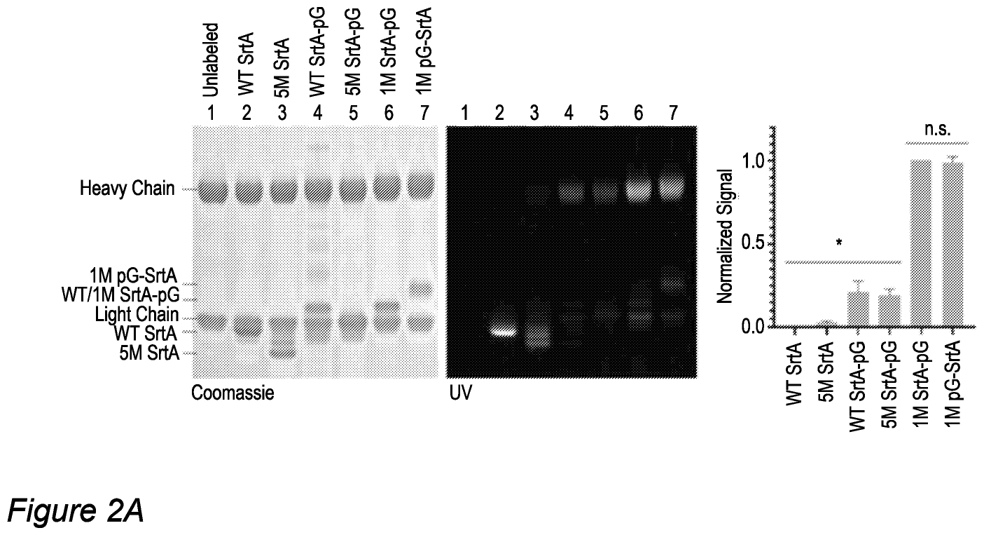

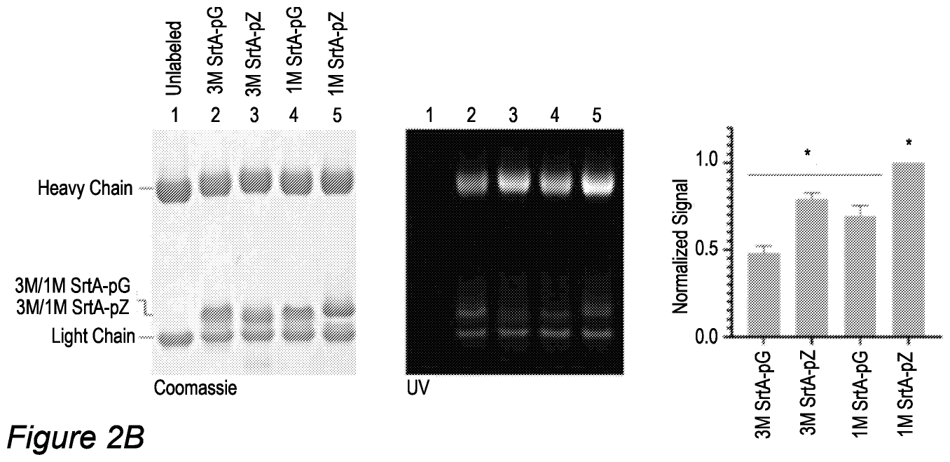

Initially, Staphylococcus aureus SrtA was tested to determine if it could be directly applied towards modifying off-the-shelf IgG antibodies. From prior studies, it has been shown that the efficiency of SrtA-catalyzed ligation reactions improves when SrtA is placed in close proximity to the protein of interest. Given this, two well-characterized and widely used SrtA variants, the wild-type SrtA (WT SrtA) and the pentamutant (P94R/D160N/D165A/K190E/K196′, referred to as 5M SrtA), were fused to a C-terminal Protein G, which facilitates binding of SrtA to the Fe region of the antibody. As shown in FIG. 2A (Lanes 2-5), both WT and 5M SrtA fused to Protein G (WT SrtA-pG and 5M SrtA-pG, respectively) demonstrated greater degree of TAMRA labeling on the heavy chain of Cetuximab compared to that of unmodified WI and 5M SrtA. This indicates that placing SrtA in close proximity to the antibody via Protein G improves the efficiency of the isopeptide ligation reaction. However, the labeling efficiency was quite low for both WT and 5M SrtA-pG, which is reasonable given that the isopeptide reaction at the lysine residue is non-canonical for SrtA. Hence, wild-type SrtA was mutated using random mutagenesis to generate a library of SrtA mutants, which were screened based on their ability to conjugate either Cetuximab or Trastuzumab. From several rounds of mutagenesis and screening, two evolved SrtA mutants were identified to improve labeling efficiency. Evolved against Cetuximab, three point mutations in SrtA-pG (T156S/D176E/D170E) (referred to as 3M SrtA-pG hereinafter) were shown to be beneficial when compared to WT and 5M SrtA-pG (not shown). Meanwhile, when evolved against Trastuzumab, a single point mutation in SrtA-pG (N127K) (referred to as 1M SrtA-pG hereinafter) drastically enhanced the conjugation activity compared to WT and 5M SrtA-pG (FIG. 2A, Lane 6). 1M pG-SrtA was also evaluated to determine if the orientation of the fusion protein affects labeling efficiency: no significant difference was found between either orientation (FIG. 2A, Lane 7). Next, 3M and 1M SrtA-pG were compared to assess which set of mutations yields the greatest conjugation activity. When labeling Cetuximab, 1M SrtA-pG was more efficient than 3M SrtA-pG (FIG. 2B). Additionally, due to enhanced solubility and elution conditions afforded by Protein Z, the fusion proteins were later re-cloned to replace Protein G with Protein Z to generate 3M and 1M SrtA-pZ. Incidentally, Protein Z also improved the labeling efficiencies of both 3M SrtA and 1M SrtA, with 1M SrtA-pZ being the most effective at labeling Cetuximab (FIG. 2B). Therefore, 1M SrtA-pZ was used to generate all Cetuximab conjugates. It is interesting to note that some labeling occurs on the light chain as well. On average, around 6% of the TAMRA labeling occurs on the light chain, which suggests the presence of some reactive lysine residue on the light chain that engages in the isopeptide ligation reaction at a lower frequency compared to on the heavy chain.

### Optimization of the Sortase-Mediated Isopeptide Ligation Reaction

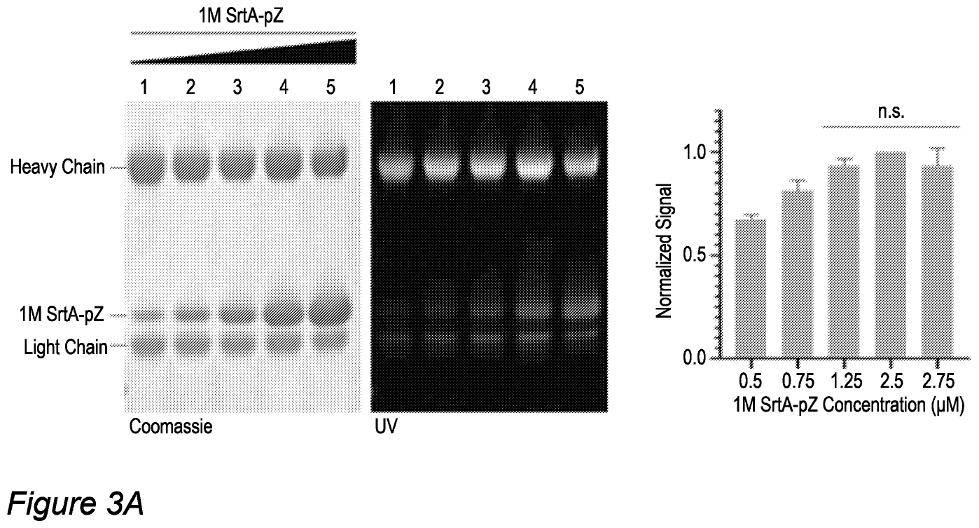

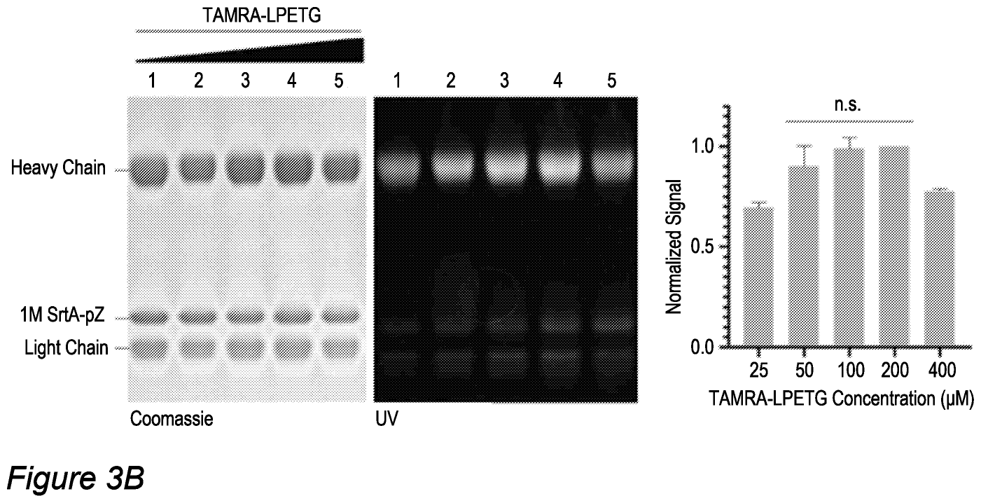

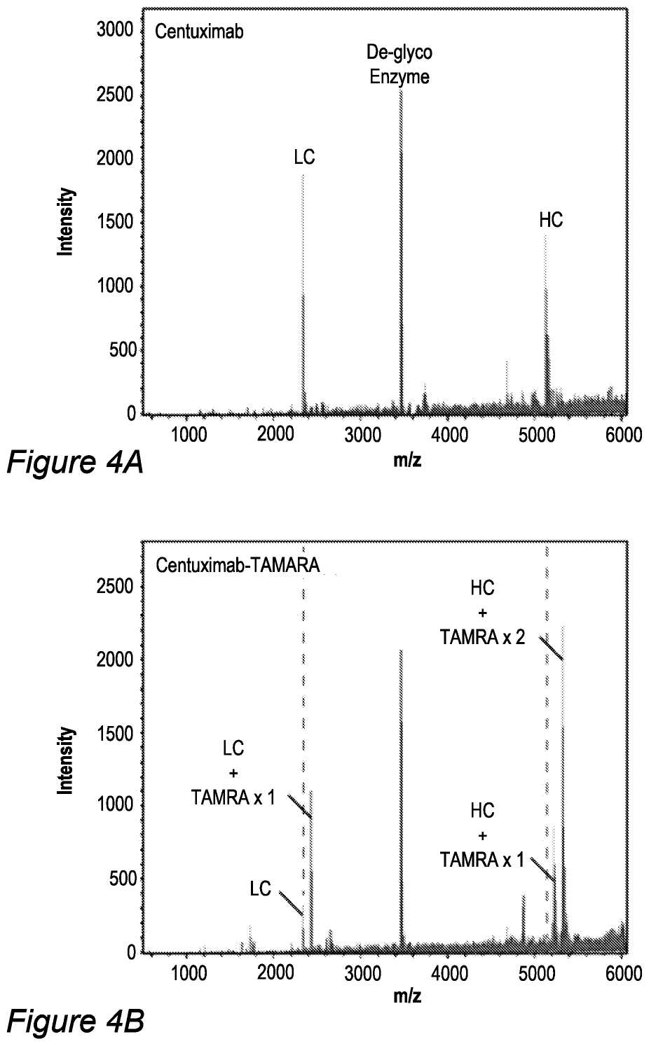

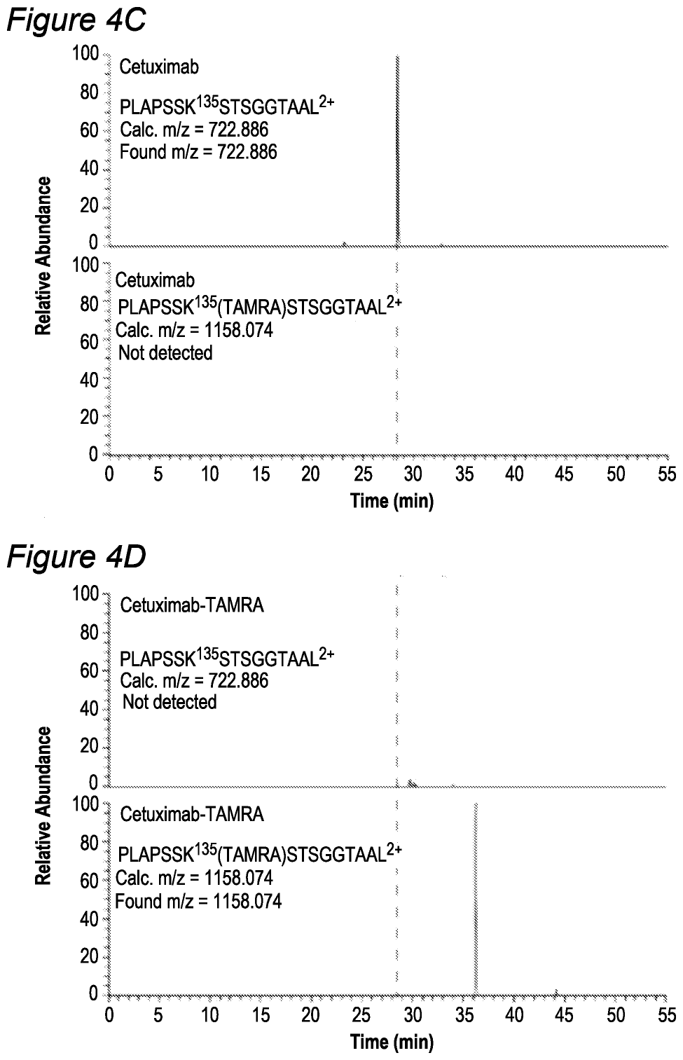

Components of the isopeptide reaction were titrated to determine the optimal conditions for labeling antibody with 1M SrtA-pZ, particularly the molar ratios of 1M SrtA-pZ and TAMRA-LPETG to antibody. First, holding all other reaction components constant, Cetuximab was incubated with increasing concentrations of 1M SrtA-pZ from 0.5-3.75 μM, which correlates to 1M SrtA-pZ/antibody molar ratios ranging from 0.4:1 to 3:1 (FIG. 3A). As 1M SrtA-pZ concentration was increased, the relative labeling efficiency on Cetuximab improved until the concentration reached 1.25 μM (1:1 molar ratio), after which the labeling efficiencies were not statistically different. Additionally, Cetuximab was incubated with increasing concentrations of TAMRA-LPETG (25-400 μM) with all other reaction components kept constant (FIG. 3B). Optimal labeling occurs at TAMRA-LPETG concentrations ranging from 50-200 μM. This suggests that the near traceless isopeptide ligation method is efficient at lower LPXTG peptide concentrations compared to current SrtA-mediated conjugation methods, which typically require a large molar excess. For all subsequent conjugation reactions, 1.25 μM of SrtA-pZ and 200 μM of TAMRA-LPETG were used.

### Quantification of the Average Drug-to-Antibody Ratio of Cetuximab-Ahx-vcTAMRA

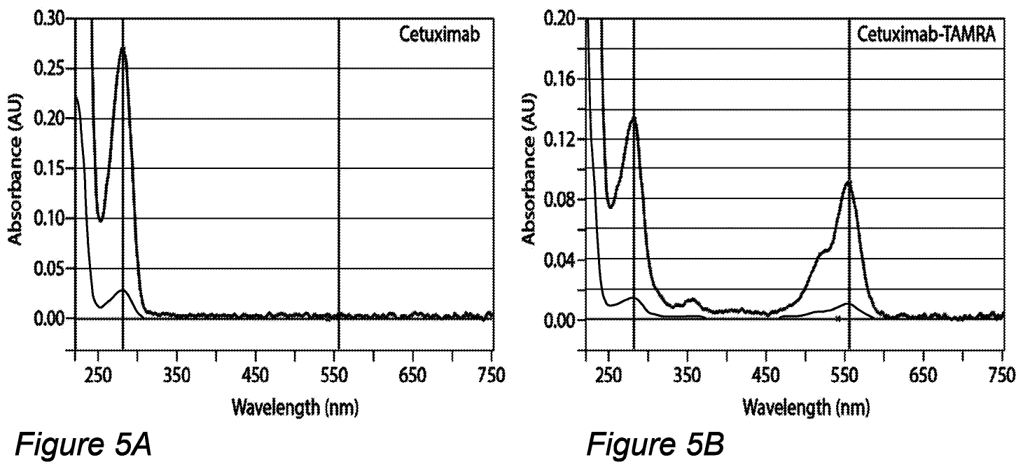

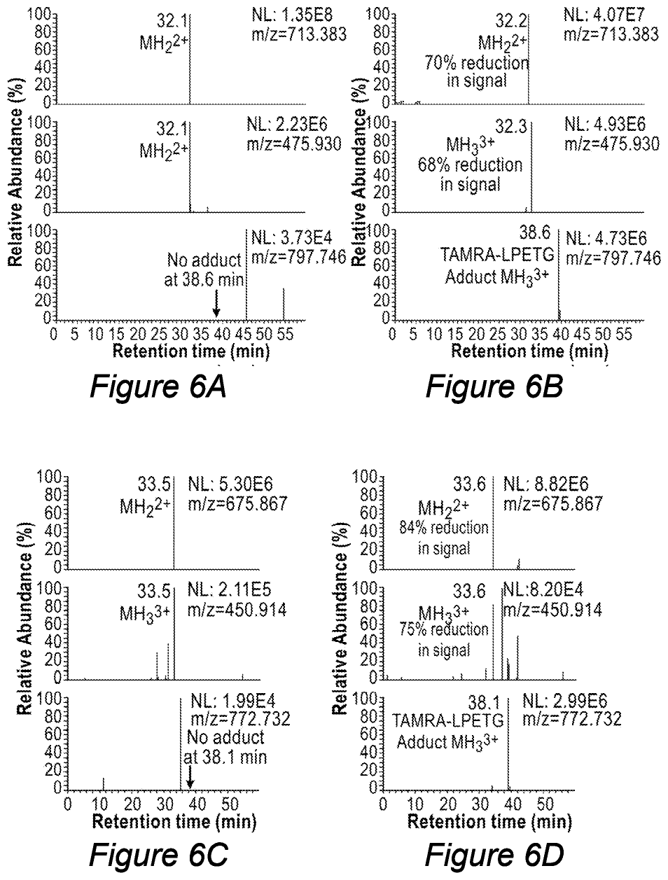

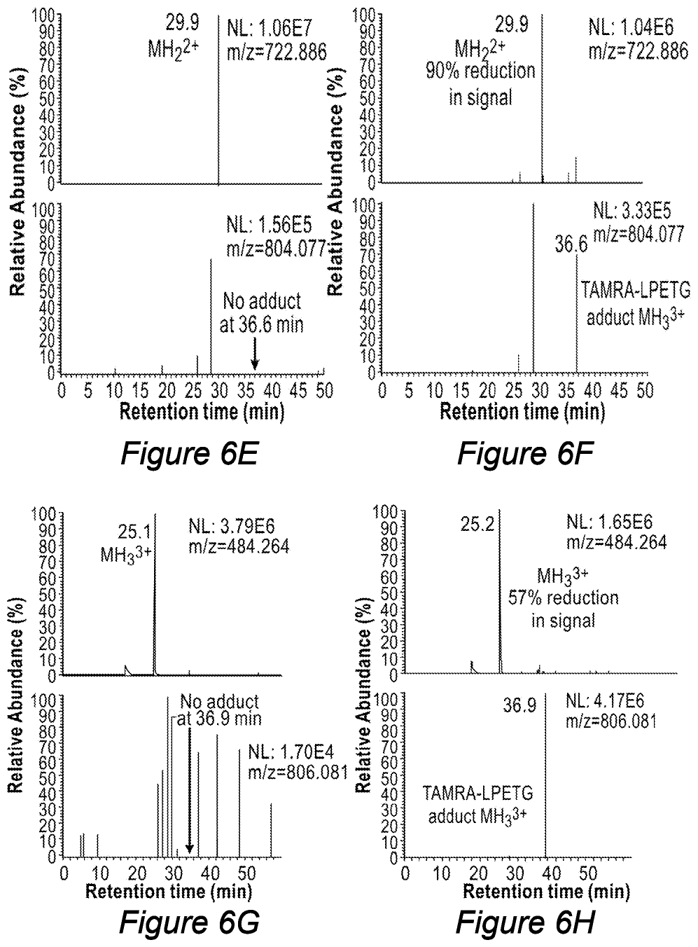

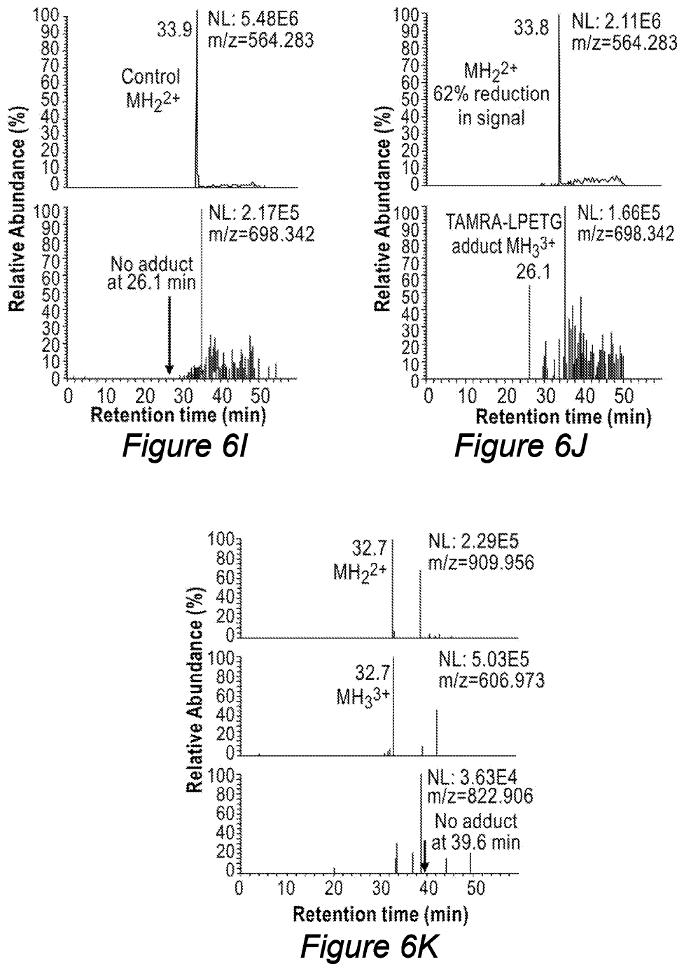

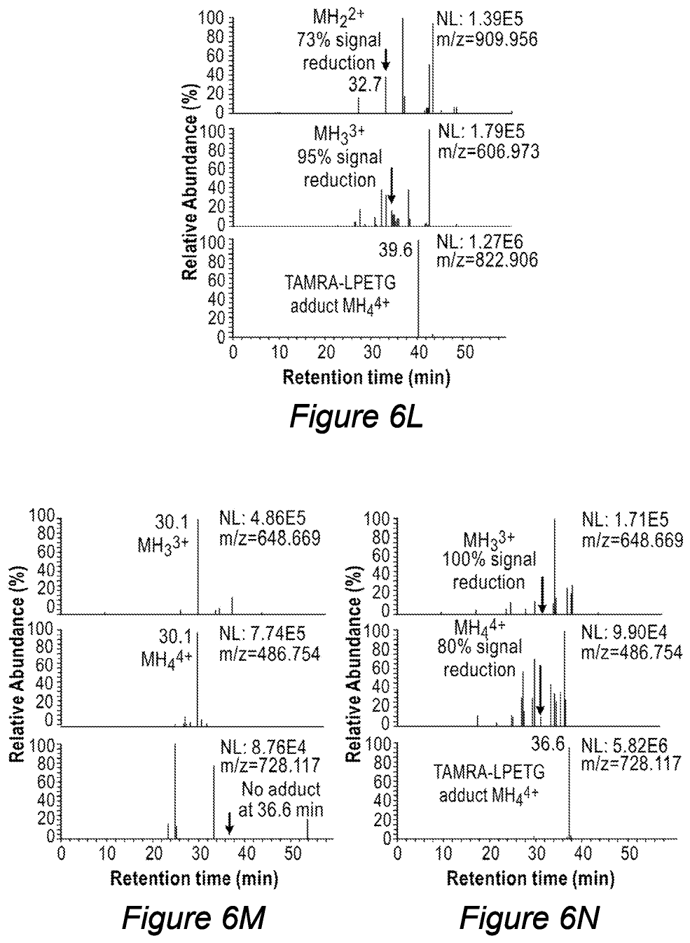

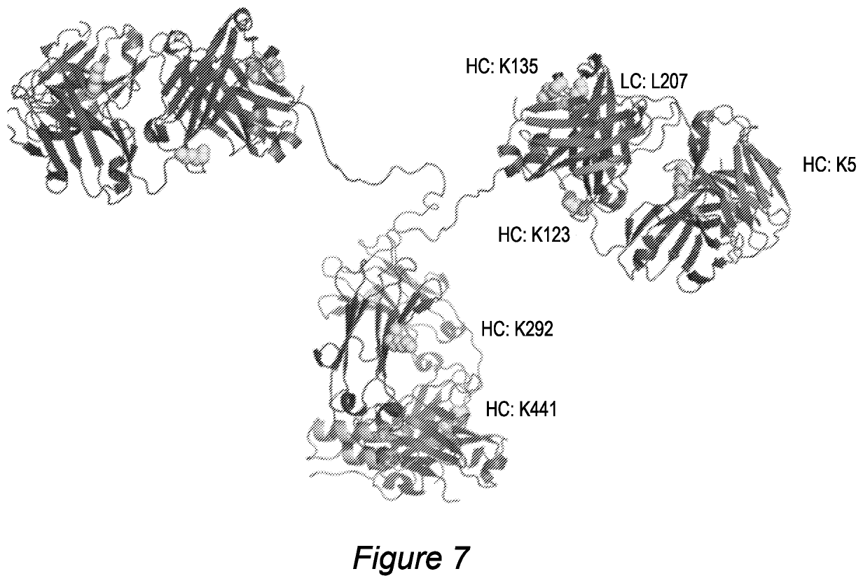

Using optimized reaction conditions, MMAE and TAMRA antibody conjugates were synthesized using a human IgG1 anti-human endothelial growth factor receptor (EGFR) antibody (Cetuximab) to generate Cetuximab-Ahx-vcMMAE and Cetuximab-Ahx-vcTAMRA. In order to determine the average DAR of Cetuximab-Ahx-vcMMAE, the average ratio of TAMRA molecules to antibody was measured for Cetuximab-Ahx-vcTAMRA, assuming that the DAR of Cetuximab-Ahx-vcMMAE is comparable. In this experiment, the DAR was measured by UV/Vis spectroscopy. The absorption spectra of cetuximab and Cetuximab-Ahx-vcTAMRA were absorption maxima for protein and TAMRA are 280 and 555 nm, respectively. From the absorption spectra, only a single absorbance peak at 280 nm is present for Cetuximab, which is unmodified so the only absorbance contribution is due to protein (FIG. 5A). For Cetuximab-Ahx-vcTAMRA, there are absorbance peaks at both 280 and 555 nm wavelengths, indicating successful TAMRA labeling on Cetuximab (FIG. 5B). Quantification of the average DAR using UV/Vis spectroscopic analysis has been previously detailed. Using this method, the average DAR was found to be 2.32.

### Assessment of Functional Binding Properties of Cetuximab-Ahx-vcMMAE

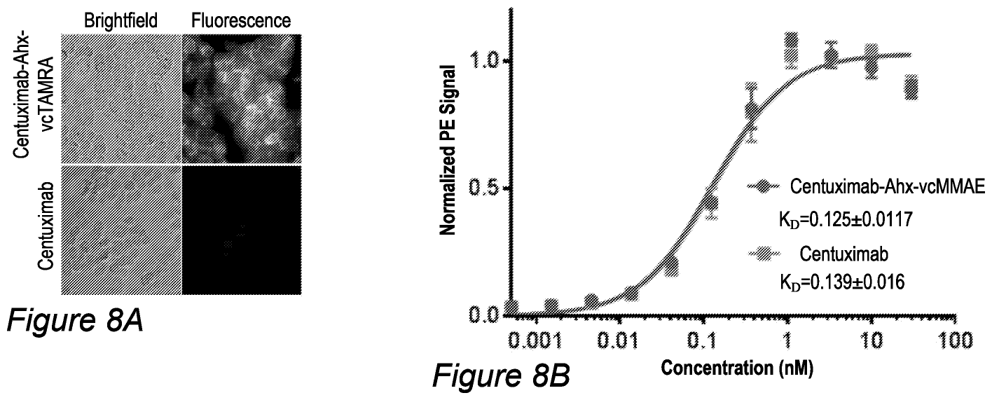

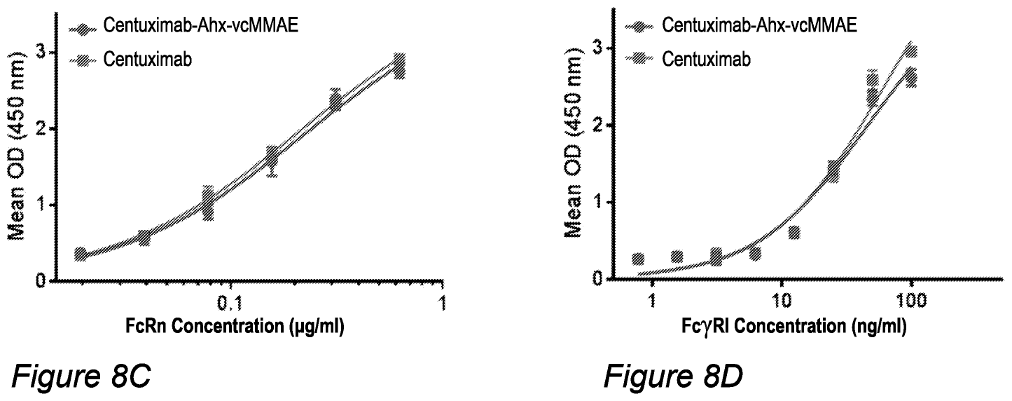

To show that antigen binding is conserved after antibody conjugation using the near traceless method, Cetuximab-Ahx-vcTAMRA and Cetuximab were applied to fixed MDA-MB 468 cells, which are EGFR⁺. Under fluorescence, positive staining on the cell surface is only observed for Cetuximab-Ahx-vcTAMRA (FIG. 8A). Moreover, the binding affinity of the conjugate was assessed by treating fixed MDA-MB 468 cells with either Cetuximab-Ahx-vcMMAE or Cetuximab, followed by a fluorescent anti-human secondary antibody. Degree of binding was measured by the fluorescent signal. The binding affinities (K_{D}) to MDA-MB 468 cells for Cetuximab and Cetuximab-Ahx-vcMMAE were very similar, indicating that the near traceless conjugation method does not interfere with the complementarity-determining regions (CDRs) of the antibody (FIG. 8B). Next, binding to the Fc region of the antibody was evaluated since the Fc region interacts with many cell-surface receptors, or Fc receptors, to activate essential functions in the immune system. For instance, the neonatal Fc receptor (FcRn) extends the half-life circulation of IgG antibodies by recycling antibodies internalized by cells back into the blood, which protects them from lysosomal degradation. In turn, FcRn binding is often desirable for IgG-based therapeutics. Since FcRn binds to IgG Fc with high affinity at acidic pH, a 96-well plate coated with Cetuximab-Ahx-vcMMAE or Cetuximab was incubated with biotinylated FcRn+B2M heterodimer at pH 6.0, followed by streptavidin-HRP and TMB for detection using absorbance. Analysis of the absorbance signal revealed equivalent FcRn binding for Cetuximab-Ahx-vcMMAE or Cetuximab, meaning that Fc-FcRn interaction is largely unaffected by the near traceless conjugation method (FIG. 8C). Along with FcRn, Fc engagement with Fc-gamma receptors play a crucial role in eliciting effector immune responses, such as antibody dependent cellular cytotoxicity (ADCC). Given this, binding to Fc-gamma receptor I (FcγRI), which is specific to IgG, was evaluated using the same assay format as the FcRn binding assay, but at physiological pH. Both Cetuximab-Ahx-vcMMAE and Cetuximab demonstrated comparable FcγRI binding (FIG. 8D). Hence, the near traceless conjugation method preserves antigen binding and Fc receptor interactions of the antibody.

### In Vitro Cytolytic Activity of Cetuximab-Ahx-vcMMAE Conjugates

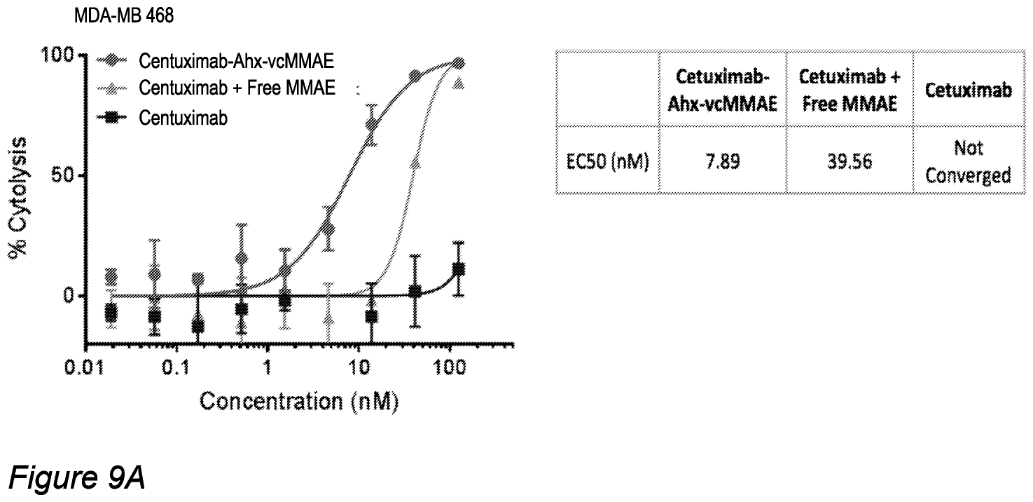

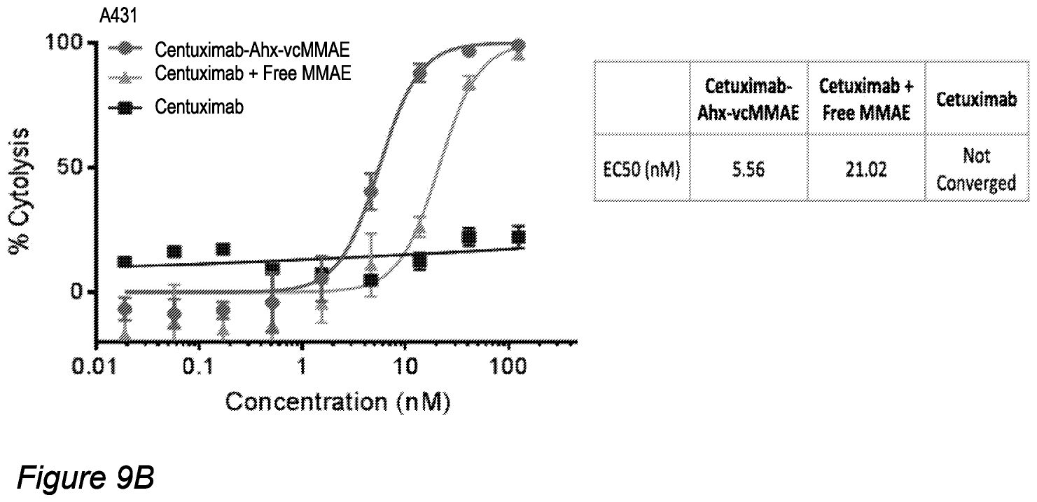

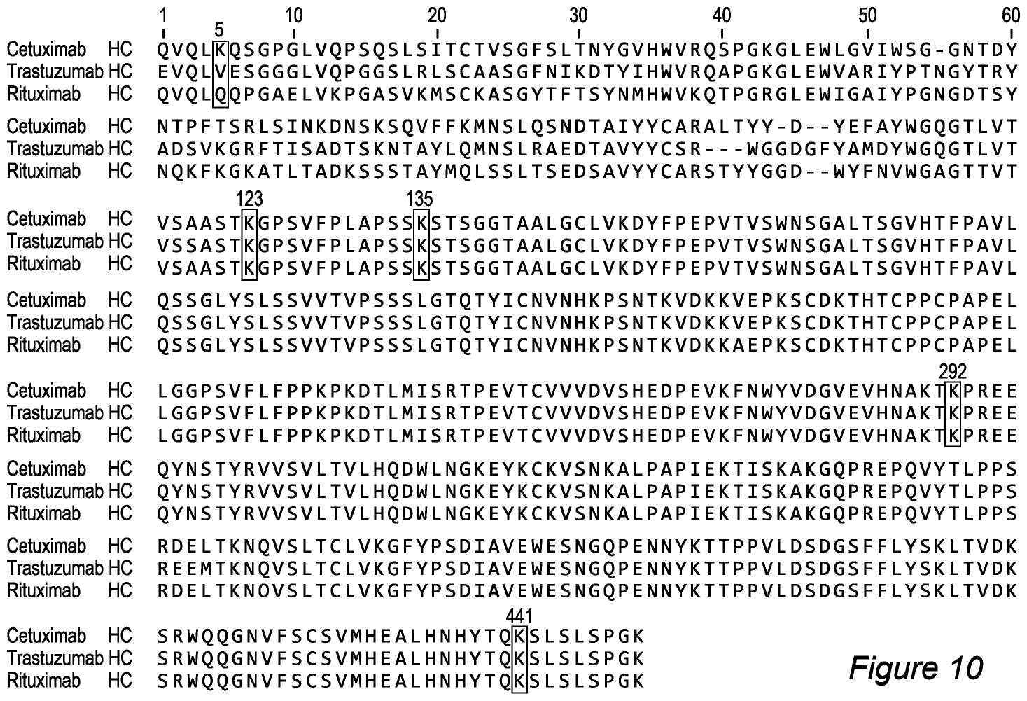

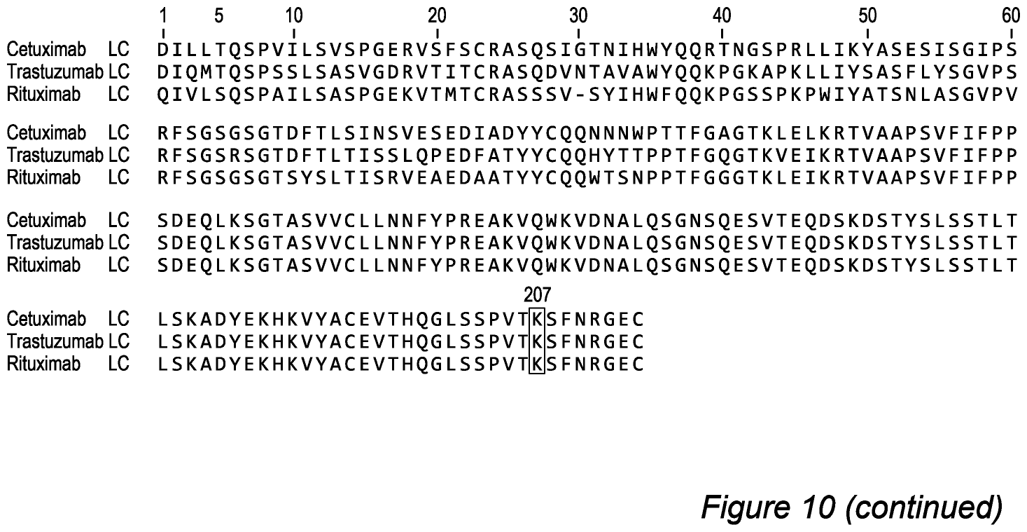

The in vitro efficacy of Cetuximab-Ahx-vcMMAE was evaluated in two EGFR⁺ cancer cell lines, MDA-MB 468 and A431. The xCELLigence Real-Time Cell Analysis (RTCA) system, a label-free method to track cell proliferation by measuring electrical impedance, was used to monitor cell viability in real time. Cells were incubated with Cetuximab-Ahx-vcMMAE, Cetuximab mixed with free MMAE (1:2 molar ratio), or Cetuximab for 96 hours. Untreated cells with PBS were included as a negative control (100% viability). At 96 hours post-treatment, cell viability data were collected, and % cytolysis was calculated for treated cells relative to untreated cells. Cetuximab alone showed little to no killing of MDA-MB 468 cells (FIG. 9A). Free MMAE mixed with Cetuximab had greater cytolytic activity with an EC₅₀ of 39.56 nM (FIG. 9A). Cetuximab-Ahx-vcMMAE had the most potent cytolytic activity with an EC₅₀ of 7.89 nM, indicating that attaching MMAE to Cetuximab enhances cytolysis since MMAE is directed into cells by antibody internalization (FIG. 9A). The same trend is seen in A431 cells, where Cetuximab-Ahx-vcMMAE had the highest cytolytic activity with an EC₅₀ of 5.56 nM (FIG. 9B). Thus, ADCs made by near traceless conjugation methods show potent in vitro efficacy.

### Applicability to Other Human IgG Antibodies

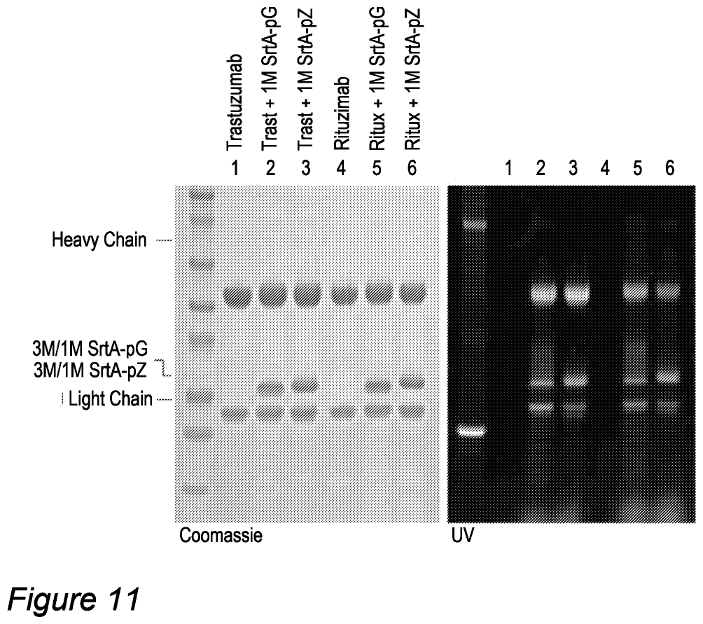

To show that other IgG antibodies can be conjugated using the near traceless method, Trastuzumab and Rituximab were labeled with TAMRA using the reaction conditions described earlier (FIG. 11). Labeling Trastuzumab seems to be quite efficient for both 1M SrtA-pG and 1M SrtA-pZ. However, the overall labeling efficiency of Rituximab is much less, especially for 1M SrtA-pZ, which is likely due to the fact that the optimal conditions for Cetuximab may not be favorable for Rituximab. In that case, reaction conditions for antibody conjugation may have to be optimized and tailored for different antibodies. Nonetheless, the near traceless conjugation method can be used to produce antibody conjugates for a variety of off-the-shelf IgGs.

### Discussion

In summary, site-specific ADC conjugation approaches yield safer and more efficacious ADCs compared to those produced by conventional conjugation methods.

A major shortcoming of current site-specific methods is that the antibody must first be modified in some way (e.g., with peptide tags, unnatural amino acids, glycan modification, etc.) to facilitate conjugation. This is disadvantageous because these modifications often interfere with the structural and functional integrity, as well as the stability, of the antibody. To address this shortfall, this disclosure describes a novel, near traceless sortase-mediated conjugation method to generate site-specific ADCs from off-the-shelf IgG antibodies. Differing from the canonical SrtA-mediated transpeptidation reaction, methods described herein employ a SrtA-catalyzed isopeptide reaction between the threonine residue on the LPXTG sortase recognition motif and a reactive lysine residue on the antibody's Fc fragment. In doing so, theoretically any LPXTG-modified compound can be ligated to an antibody without having to modify it first. This Example was divided into two primary objectives: (1) optimize the labeling efficiency of near traceless conjugation methods, and (2) apply these methods to engineer functional site-specific ADCs.

First, the near traceless conjugation method was optimized to maximize the labeling efficiency of the isopeptide reaction. In this study, through fusion to Protein G, SrtA was found to more efficiently catalyze the isopeptide ligation reaction when it was in close proximity to the reactive lysine residue on the Fc fragment. In addition, SrtA was directly evolved to identify point mutations that improve its ability to catalyze the isopeptide reaction and facilitate conjugation of LPXTG-modified molecules to different antibodies, particularly Cetuximab and Trastuzumab. After several rounds of random mutagenesis and screening, two superior mutation schemes were found: a tri-mutant SrtA (3M) and a single mutant SrtA (1M). For the isopeptide ligation reaction, 3M and 1M SrtA-pG more efficiently labeled Cetuximab by several fold compared to wild-type (WT) and pentamutant SrtA (5M) even when fused to Protein G. Both WT and 5M SrtA have been extensively studied and employed for protein conjugation applications using the canonical ligation reaction. Specifically, compared to WT SrtA, 5M SrtA has been shown to display 120-fold greater LPXTG coupling and 20-fold greater resolution by GGG. However, in the non-canonical isopeptide reaction, 5M SrtA-pG has no advantage over WT SrtA-pG in terms of labeling efficiency. This could be due to the fact that the thioester acyl-enzyme intermediate involving 5M and WT SrtA-pG are inefficiently resolved by the ε-amine of the lysine residue on the antibody. Later in these studies, Protein G was replaced with Protein Z, because Protein Z improved the solubility of the fusion protein and made removal of SrtA-pZ from the antibody more convenient. Interestingly, 3M and 1 M SrtA-pZ fusions exhibited even greater TAMRA labeling on Cetuximab compared to their Protein G counterparts, with 1M SrtA-pZ having the best labeling efficiency. As a result, 1M SrtA-pZ was then used for all conjugation reactions. Protein G and Protein Z have slightly different binding domains on the Fc region of IgG antibodies; thus, the binding domain for Protein Z may provide some advantage in the isopeptide reaction that provides SrtA with greater access to the reactive lysine of interest. In efforts to further optimize the isopeptide ligation reaction, it was found that a 1:1 molar ratio of 1M SrtA-pZ to antibody, as well as TAMRA-LPETG concentrations as low as 50 μM, is sufficient for achieving maximum labeling. This likely can be attributed to the fact that 1M SrtA-pZ exhibits a high catalytic efficiency. As a result, isopeptide ligation is effective at lower LPXTG peptide concentrations, which is advantageous since a large molar excess of LPXTG-modified compounds that is often necessary for most SrtA-mediated conjugation methods is not required. In turn, expensive LPXTG-modified compounds, or those in limited quantities, can be conserved using this method. Furthermore, this method is beneficial in that antibody conjugate yield is not limited by protein expression. The only component in the reaction that requires expression is 1M SrtA-pZ, which can be purified in large quantities (˜30-50 mg per L culture), while all other components are commercially available.

Employing the optimized near traceless conjugation method, site-specific ADCs were generated by directly conjugating MMAE to unmodified Cetuximab (Cetuximab-Ahx-vcMMAE). ADCs with an average DAR of 2.32 were successfully produced. Characterization of the in vitro binding properties of the ADCs indicated that near traceless conjugation preserves antigen binding to Cetuximab. Moreover, Fe interactions with Fe receptors, such as FcRn and FcγRI, remained intact after conjugation. FcRn binding is important due to its role in extending the half-life circulation of IgG antibodies in vivo, which is desirable for many antibody therapeutics, such as ADCs. Meanwhile, FcγRI binding plays an essential role in activating the immune response, particularly through cell effector functions, such as ADCC. As a result, this conjugation method could be utilized to produce ADCs that maintain these therapeutic functionalities. The in vitro cytolytic potency of the AIX's was also evaluated for two EGFR cancer cell lines, MDA-MB 468 and A431. In both target cell lines, the ADCs demonstrated superior cytolytic activity compared to conditions where the cells were treated with Cetuximab mixed with free MMAE or Cetuximab alone. Finally, the versatility of the near traceless conjugation method was explored by investigating whether or not other human IgG antibodies could be labeled using this approach. Trastuzumab and Rituximab were both successfully labeled with TAMRA, but at a lower degree for Rituximab, which could be a result of working in reaction conditions that are not universally favorable for all antibodies. As such, reaction conditions for conjugating different antibodies may need to be optimized on an individual basis to achieve maximal labeling efficiency. Based on these findings, the near traceless conjugation method can be employed to produce functional, site-specific ADCs directly from off-the-shelf IgGs, which is quicker and more convenient than other site-specific conjugation approaches. Given its versatility, this method can also be explored as a general antibody conjugation platform technology to attach a variety of compounds (e.g., fluorophores, chemical handles for click chemistry, proteins, etc.) in addition to drug payloads.

The exact lysine residue(s) involved in the isopeptide ligation reaction can be further investigated. Based on TAMRA labeling, a majority of the conjugated TAMRA was found on the heavy chain of the antibody, which suggests that the reactive lysine residue(s) of interest are located in the CH2 and CH3 domains. This makes intuitive sense since SrtA is attached to the Fc fragment via Protein Z so SrtA is more likely to react with lysines that are closer in proximity. However, on average, 6% of the TAMRA labeled on the antibody was found on the light chain. This indicates that there is some reactive lysine residue present on the light chain that is involved in the isopeptide reaction. The involved lysines of interest can be identified via peptide mapping by mass spectrometry. Additionally, lysines at select positions on the antibody can be systematically knocked down via alanine mutagenesis. After which, each antibody mutant is screened to determine whether or not that knockdown can be labeled. If the antibody mutant cannot be labeled, the lysine that was mutated in the antibody is essential for conjugation using this method. In this study, the average DAR was quantified using IV/Vis spectroscopy, which provides a good estimation of the average DAR. However, it is not the most precise method. Also, this approach is incapable of assessing the drug load distribution of the ADC's so the homogeneity of the ADC population is unknown. A more robust method, such as liquid chromatography coupled with electrospray ionization mass spectrometry (LC-ESI-MS), is used to more precisely quantify the DAR and assess homogeneity. Furthermore, while in vitro studies revealed potent ADC activity, in vivo studies in rodent xenograft models are conducted to assess the anti-tumor efficacy, toxicity, and stability of the ADCs compared to those prepared by conventional conjugation techniques. Lastly, other applications of this near traceless conjugation method are explored to determine its versatility as a platform technology for antibody conjugation. To expand its generality as an antibody conjugation method, this method is investigated further to show that antibodies from different species and isotypes can be labeled using this approach. One potential application of this method would be to attach other proteins to antibodies to create novel protein fusions, such as bispecific antibodies which have become increasingly popular in cancer immunotherapy research in recent years. Due to its complexity, there are many challenges associated with expressing recombinant bispecific antibodies. Hence, by covalently linking small antibody proteins, such as affibodies, to defined sites on off-the-shelf IgGs using methods described herein, homogenous bispecific antibodies can be conveniently generated without issues regarding protein expression and heterogeneity.

In conclusion, this Example describes innovative, near traceless sortase-mediated conjugation methods that were developed to conveniently produce site-specific ADCs from off-the-shelf IgG antibodies. A novel point mutation in SrtA was identified to significantly improve the catalytic efficiency of SrtA in the isopeptide ligation reaction. To validate this conjugation approach, site-specific ADCs were successfully engineered from unmodified Cetuximab and then characterized in in vitro studies. These ADCs were shown to have an average DAR of ˜2, conserved antigen binding and Fe-receptor interactions, and strong in vitro cytolytic potency. The near traceless methods described herein have demonstrated great potential as a versatile antibody conjugation technology.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A composition for producing an antibody conjugate, comprising a fusion protein that comprises a sortase and an antibody binding domain that binds to an antibody.

2. The composition of claim 1, wherein the sortase is sortase A or a variant thereof.

3. The composition of claim 2, wherein the sortase A has one or more of point mutations selected from T156S, D176E, and D170E.

4. The composition of claim 2, wherein the sortase A has an N127K point mutation.

5. The composition of claim 1, wherein the antibody binding domain is protein G, protein A, a protein G variant, a protein A variant, or a subdomain of protein G or protein A.

6. The composition of claim 1, wherein the antibody binding domain comprises a protein selected from a Protein G HTB1 domain, a Protein Z domain, a Protein A, a Protein G, a Protein L, a Protein LG, a Protein LA, a Protein A/G, or an Fc-binding peptide, such as Fc-III, Fc-III-4C, APAR, PAM, FcBP-2, RRGW, KHRFNKD, or a functional sub-domains thereof.

7. A method of producing an antibody conjugate, comprising:
a) contacting an antibody with a fusion protein comprising a sortase and an antibody binding domain, wherein the antibody binding domain binds to the antibody; and
b) linking a peptide that comprises a sortase recognition motif to the antibody via a ligation mediated by the sortase, wherein an isopeptide bond is formed between the peptide and a lysine on the antibody, thereby forming an antibody conjugate comprising the peptide.

8. The method of claim 7, wherein the sortase is sortase A or a variant thereof.

9. The method of claim 8, wherein the sortase A has one or more of point mutations selected from T156S, D176E, and D170E.

10. The method of claim 8, wherein the sortase A has an N127K point mutation.

11. The method of claim 7, wherein the antibody binding domain is protein G, protein A, a protein G variant, a protein A variant, or a subdomain of protein G or protein A.

12. The method of claim 7, wherein the antibody binding domain comprises a protein selected from a Protein G HTB1 domain, Protein Z domain, Protein A, Protein G, Protein L, Protein LG, Protein LA, Protein A/G, or an Fc-binding peptide, such as Fc-III, Fc-III-4C, APAR, PAM, FcBP-2, RRGW, KHRFNKD, or a functional sub-domains thereof.

13. The method of claim 7, wherein the peptide further comprises a fluorescent dye, a hapten, a polymer, a contrast agent, a radionuclide, a chelated metal, a therapeutic agent, a sensitizer, an oligonucleotide, or combinations thereof.

14. The method of claim 7, wherein the sortase recognition motif has a sequence selected from SEQ ID NOs: 1-21.

15. A method of producing an antibody conjugate, comprising:
a) contacting an antibody with a fusion protein comprising a sortase and an antibody binding domain, wherein the antibody binding domain binds to the antibody, and the antibody comprises a sortase recognition motif near the C-terminus of one or both of its heavy chain and light chain; and
b) linking a peptide comprising a N-terminus glycine to the antibody via a ligation mediated by the sortase, wherein a peptide bond is formed between the peptide and the sortase recognition motif on the antibody, thereby forming an antibody conjugate comprising the peptide.

16. The method of claim 15, wherein the sortase is sortase A or a variant thereof.

17. The method of claim 16, wherein the sortase A has one or more of point mutations selected from T156S, D176E, and D170E.

18. The method of claim 16, wherein the sortase A has an N127K point mutation.

19. The method of claim 14, wherein the antibody binding domain is protein G, protein A, a protein G variant, a protein A variant, or a subdomain of protein G or protein A.

20. The method of claim 14, wherein the peptide further comprises a fluorescent dye, a hapten, a polymer, a contrast agent, a radionuclide, a chelated metal, a therapeutic agent, a sensitizer, an oligonucleotide, or combinations thereof.

21. The method of claim 14, wherein the sortase recognition motif has a sequence selected from SEQ ID NOs: 1-21.

22. An isolated polynucleotide encoding a fusion protein comprising a sortase and an antibody binding domain that binds to an antibody.

23. The isolated polynucleotide of claim 22, wherein the sortase is sortase A or a variant thereof.

24. The isolated polynucleotide of claim 23, wherein the sortase A has one or more of point mutations selected from T156S, D176E, and D170E.

25. The isolated polynucleotide of claim 23, wherein the sortase A has an N127K point mutation.

26. The isolated polynucleotide of claim 22, wherein the antibody binding domain is protein G, protein A, a protein G variant, a protein A variant, or a subdomain of protein G or protein A.

27. The isolated polynucleotide of claim 22, wherein the antibody binding domain comprises a protein selected from a Protein G HTB1 domain, a Protein Z domain, a Protein A, a Protein G, a Protein L, a Protein LG, a Protein LA, a Protein A/G, or an Fc-binding peptide, such as Fc-111, Fc-III-4C, APAR, PAM, FcBP-2, RRGW, KHRFNKD, or a functional sub-domains thereof.

28. A vector comprising the isolated polynucleotide of claim 22.

29. A cell comprising the vector of claim 28.
